(19) United States
(12) Patent Application Publication
(10) Pub. No.: US 2025/0011438 A1
(43) Pub. Date: Jan. 9, 2025

(54) Antibody Binding to Tie2 and Use Thereof

(71) Applicant: Institute for Basic Science, Daejeon (KR)

(72) Inventors: Gou Young Koh, Daejeon (KR); Jeomil Bae, Daejeon (KR); Jaeryung Kim, Daejeon (KR)

(73) Assignee: Institute for Basic Science, Daejeon (KR)

(21) Appl. No.: 18/667,700

(22) Filed: May 17, 2024

Related U.S. Application Data
(62) Division of application No. 17/111,413, filed on Dec. 3, 2020, now Pat. No. 12,024,562.
Application No. 17/111,413 is a continuation of application No. PCT/KR2019/006820, filed on Jun. 5, 2019.
(60) Provisional application No. 62/682,042, filed on Jun. 7, 2018.

(30) Foreign Application Priority Data
Jun. 5, 2019 (KR) 10-2019-0066622

(51) Int. Cl. C07K 16/28; A61K 31/713; A61K 39/00; A61K 39/395; A61K 45/06; A61P 27/06; C12N 5/10; C12N 15/63

(52) U.S. Cl. CPC C07K 16/2863; A61K 31/713; A61K 39/3955; A61K 45/06; A61P 27/06; C12N 5/10; C12N 15/63; A61K 2039/505; C07K 2317/24; C07K 2317/34; C07K 2317/565; C07K 2317/92; C07K 2317/94

(57) ABSTRACT

The present invention refers to an antibody against Tie-2 or antigen-binding fragment thereof, a nucleic acid encoding the same, a vector comprising the nucleic acid, a cell transformed with the vector, a manufacturing method of the antibody or antigen-binding fragment thereof, and a composition for preventing or treating angiogenic diseases, which comprises the antibody or antigen-binding fragment thereof.

This application is a divisional of application Ser. No. 17/111,413, filed December 3. 2020, which is a continuation of International Patent Application No. PCT/KR2019/006820, filed Jun. 5, 2019, which claims the benefit of Korean Patent Application No. 10-2019-0066622, filed Jun. 5, 2019, and U.S. Provisional Patent Application No. 62/682,042, filed Jun. 7, 2018, all of which are incorporated herein by reference in their entirety for all purposes.

The present application contains a Sequence Listing which has been submitted electronically in XML format. Said XML copy, created on Apr. 23, 2023, is named “01262-0003-01US_ST26.xml” and is 69,131 bytes in size. The information in the electronic format of the sequence listing is incorporated herein by reference in its entirety.

### FIELD OF THE INVENTION

The present invention relates to an antibody against Tie-2 or an antigen-binding fragment thereof, a nucleic acid encoding the same, a vector containing the nucleic acid, a cell transformed with the vector, a method for preparing the antibody or antigen-binding fragment thereof, and a pharmaceutical composition to prevent or treat angiogenic diseases.

### DESCRIPTION OF THE BACKGROUND

Angiogenesis occurs dynamically by a variety of regulatory factors during the development, growth, maintenance, and homeostasis of an organism. Blood vessels newly formed in this process act as transport channels for various biomaterials such as nutrients, oxygen, and hormones in the surrounding cells. Functionally and structurally abnormal blood vessels are the direct or indirect cause for the initiation and progression of various diseases. Tumor blood vessels aggravate hypoxia due to their defective function and structure, resulting in tumor progression and metastasis to other tissues, and also in the poor delivery of anticancer drugs into the core of the tumor mass. Defective blood vessels are also found in other various diseases and conditions, in addition to cancer. Examples thereof include various ocular diseases (e.g., diabetic macular edema, wet age-related macular degeneration), viral infections, and acute inflammatory responses such as sepsis. Thus, if a therapeutic agent capable of normalizing pathologic blood vessels is available, it can be applied to the treatment of various patients with vascular abnormalities.

The angiopoietin family plays an important role in the formation and maintenance of blood vessels, and is comprised of four angiopoietins (Ang1, Ang2, Ang3, and Ang4). Angiopoietin-1 (Ang1) binds to the Tie2 receptor present on the surface of vascular endothelial cells to phosphorylate and activate Tie2 receptor, resulting in stabilization of blood vessels. On the other hand, angiopoietin-2 (Ang2) binds to the Tie2 receptor, but acts as an antagonist to induce inactivation of the Tie2 receptor, resulting in destabilization of blood vessels and leakage of blood vessels. It was reported that the expression level of Ang2 is highly increased in the blood of cancer patients, ocular diseases, viral and bacterial infections and inflammatory diseases (Saharinen P et al., 2017, Nature Review Drug Discovery). However, Ang2 is also known to act as an agonist to induce activation of the Tie2 receptor in several processes, including lymphatic tube formation and maintenance, and thus it is believed that Ang2 performs various functions depending on the context.

Up to now, development and clinical test of various Anti-Ang2 antibodies have been intensively focused by many biopharmaceutical companies (e.g., U.S. Pat. Nos. 7,658,924, and 8,987,420). These Ang2 antibodies are shown to inhibit the binding of Ang2 to Tie2 and these Ang2 neutralizing effect was eventually shown to hinder the formation of new blood vessel. The anti-angiogenic and anti-cancer activities of these anti Ang2-antibodies have been demonstrated in many preclinical models, and diverse anti-Ang2 antibodies are being clinically tested in various cancer patients. However, their anti-cancer efficacy has been demonstrated to be insufficient. For example, Phase 3 clinical trials conducted by Amgen showed that the anti-cancer efficacy of the Ang2 antibody in ovarian cancer patients was insignificant (Marth C et al., 2017, Eur. J. Cancer). In addition to cancer models, a Ang2 neutralizing antibody, Nesvacumab, was tested in ocular patients, however it failed to improve the efficacy of Eylea (anti-VEGF) in the clinical phase 2 combo-study.

In contrast to the above-mentioned Ang2 neutralization approach, direct Tie2 activation has been also considered as an alternative approach to inhibit angiogenesis and suppress vascular permeability. Recombinant proteins, which bind directly to the Tie2 receptor and induce phosphorylation and activation of Tie2, have also been developed and tested in many preclinical cancer and ocular models. Examples thereof include COMP-Angl (Cho et al., 2004, PNAS) and Vasculotide (David S et al., 2011, Am J Physiol Lung Cell Mol Physiol). Although these agents showed anti-angiogenic and anti-permeability activity, these have very short half-life and unstable physicochemical properties. In addition, a small molecule compound (AKB-9778) was developed as an inhibitor for a phosphatase, VE-PTP which inactivate Tie2 by removing a phosphate group from phosphorylated Tie2 (Goel S, 2013, J Natl Cancer Inst). This compound indirectly increases Tie2 activity by inhibiting VE-PTP, although it has the disadvantage of activating other receptors as well (Frye M, 2015, J Exp. Med, Hayashi M, 2013, Nature Communication, Mellberg S et al., 2009, FASEB J.). In addition, agonistic Tie2 antibodies have been developed (U.S. Pat. No. 6,365,154B1, US20170174789A1). These antibodies increased the survival of endothelial cells and inhibited the vascular leakage. Interestingly, herbal extracts were shown to activate Tie2 activity and claimed to be used as skin care cosmetics (for example, JP2011102273A, JP2018043949A, JP2015168656A).

Under this technical background, the inventors of the present application made an effort to develop antibodies which specifically bind to Tie2. As a result, the inventors developed Tie2 antibodies that bind with affinity, and confirmed that these Tie2 antibodies can play a role as a therapeutic agent for angiogenic disease by inducing the phosphorylation and the activation of the Tie2 receptor, and completed the present invention.

### SUMMARY OF THE INVENTION

The object of the present invention is to provide a new anti-Tie2 antibody or antigen-binding fragment thereof.

Another object of the present invention is to provide a nucleic acid encoding the antibody or antigen-binding fragment thereof.

Another object of the present invention is to provide a vector containing the nucleic acid, a cell transformed with the vector and a method for producing the same.

Another object of the present invention is to provide a composition comprising the antibody or antigen-binding fragment thereof for preventing or treating angiogenic diseases.

Another object of the present invention is to provide a composition for comprising the antibody or antigen-binding fragment thereof and for co-administration with other therapeutic agents for angiogenic diseases.

In order to achieve the above object, the present invention provides anti-Tie2 antibody or antigen-binding fragment thereof that binds to the Ig3-FNIII (1-3) domain comprising the sequence of SEQ ID NO: 2.

Specifically, the present invention provides also anti-Tie2 antibody or antigen-binding fragment thereof comprising a heavy chain variable region comprising heavy chain CDRs comprising the amino acid sequences of SEQ ID: 3 to 5, a light chain variable region comprising light chain CDRs comprising the amino acid sequences of SEQ ID: 6 to 8; a heavy chain variable region comprising heavy chain CDRs comprising the amino acid sequences of SEQ ID: 13 to 15, a light chain variable region comprising light chain CDRs comprising the amino acid sequences of SEQ ID: 16 to 18; a heavy chain variable region comprising heavy chain CDRs comprising the amino acid sequences of SEQ ID: 23 to 25, a light chain variable region comprising light chain CDRs comprising the amino acid sequences of SEQ ID: 26 to 28: a heavy chain variable region comprising heavy chain CDRs comprising the amino acid sequences of SEQ ID: 33 to 35, a light chain variable region comprising light chain CDRs comprising the amino acid sequences of SEQ ID: 36 to 38; or a heavy chain variable region comprising heavy chain CDRs comprising the amino acid sequences of SEQ ID: 43 to 45, a light chain variable region comprising light chain CDRs comprising the amino acid sequences of SEQ ID: 46 to 48.

The present invention provides also a nucleic acid encoding the antibody or antigen-binding fragment thereof.

The present invention provides also a vector encoding the nucleic acid.

The present invention provides also a cell transformed with the vector.

The present invention also provides a manufacturing method of the antibody or antigen-binding fragment thereof including the next steps: (a) the step for culturing the cell thereof; and (b) the recovering step for the antibody or antigen-binding fragment thereof from the cell.

The present invention also provides a composition for preventing or treating angiogenesis-related diseases including the antibody or antigen-binding fragment thereof as an active ingredient.

The present invention also provides a composition for co-administration with other angiogenic disease therapeutic agent including the antibody or antigen-binding fragment thereof.

### BRIEF DESCRIPTION OF THE DRAWINGS

FIG. 1 shows analysis of Akt phosphorylation induced by anti-Tie2 antibodies. HUVECs were serum-starved for 6 hr and incubated with COMP-Ang1 (CA1, 0.5 μg/ml) or anti-Tie2 antibodies (11C4, 4A4, 3B2, 3E12 and 3H7) for 30 min. Cell lysates were subjected to SDS-PAGE/Western blotting and blots were probed with anti-phospho-Akt (S473) or anti-Akt antibody.

FIG. 2 shows analysis of dose-dependent Tie2 phosphorylation (pTie2) triggered by anti-Tie2 antibody 3H7. The capability of 3H7 antibody to induce Tie2 phosphorylation was investigated by immunoprecipitation and Western blotting analysis. Serum-starved HUVECs were incubated for 30 min with various concentrations of 3H7 antibody. As a control, HUVECs were incubated with Ang2/control Ab mixture. The cell lysates were subjected to immunoprecipitation with anti-Tie2 antibody, followed by SDS-PAGE/Western blotting analysis. Tie2 phosphorylation was probed by mouse anti-phospho-tyrosine (pY) antibody 4G10.

FIG. 3 shows Tie2 endocytosis and FOXO1 translocation triggered by anti-Tie2 antibody 3H7. HUVECs were serum starved for 6 hr and were incubated with 3H7, or Ang2 (A2) together with Control anti-Ang2 antibody (Control Ab, 1 ug/ml) for 30 min. After fixation, HUVECs were stained with DAPI, anti-Tie2 antibody, and anti-FOXO1 antibody to investigate the localization of clustered-Tie2 receptor, and FOXO1. Arrowheads indicate the endocytosed Tie2 receptor by 3H7.

FIGS. 4A-4B show inhibition of VEGF or TNF-a induced-vascular permeability by 3H7. HUVECs were seeded on transwell chamber and grown for 3 days. At 100% confluence, HUVECs were pre-treated with Ang2 (A2, 1 ug/ml), Ang2 together with Control Ab (A2+Control Ab, 1 ug/ml), or 3H7 (1 ug/ml) for 30 min and treated with VEGF (500 ng/ml) for 45 min (A) or TNF-a (100 ng/ml) for 22 hr (B) into the upper chamber. Vascular permeability was assessed by measuring FITC fluorescence in the lower chamber after adding FITC-dextran for 20 min into the upper chamber. Values are mean±SD. *p<0.05, **p<0.01, ***p<0.001 by one-way ANOVA.

FIG. 5 shows heat map representing the significant difference region in deuterium uptake when Tie2 alone or Tie2/3H7 complex was tested in hydrogen/deuterium (H/D) exchange-mass spectrometry analysis. Heat map of deuterium uptake per residue at 0.333, 10, 60, and 240 min for hTie2 antigen was generated in the absence or presence of anti-Tie2 an tibody 3H7 using the data of H/D exchange-mass spectrometry. The heat map analysis of the deuterium uptake difference indicated that the epitopes to which antibody 3H7 binds are residues 633 to 644 (SEQ ID NO: 1, TLSDILPPQPEN) and 713-726 (SEQ ID NO: 1, FAENNIGSSNPAFS) of hTie2 (Table 13). The complete sequence shown in FIG. 5 is residues 583-738 of SEQ ID NO: 1. Grey scale indicates the percentage of H/D exchange per residue between the Tie2 alone and Tie2/3H7 mixtures at each individual time.

FIG. 6 is the schematic showing 3H7-binding epitope on Tie2. Anti-Tie2 antibody 3 H7-binding epitope (black) on the FNIIIc of the hTie2 antigen, analyzed by H/D exchange-mass spectrometry, was visualized in the image of hTie2 FNIII (1-3) crystal structure (PDB: 5UTK) using PyMol™ software. The sequences shown in FIG. 6 are residues 633 to 644 of SEQ ID NO: 1 (TLSDILPPQPEN) and residues 713-726 of SEQ ID NO: 1 (FAENNIGSSNPAFS).

FIG. 7 shows phosphorylation of Akt (pAkt) induced by humanized anti-Tie2 antibodies. Serum-starved HUVECs were incubated for 30 min with humanized anti-Tie2 antibodies. Thereafter, the cell lysates were subjected to SDS-PAGE/Western blotting and blots were probed with anti-phospho-Akt (S473) or anti-Akt antibody.

FIGS. 8A-8F show increased area of SC and decreased IOP by humanized Tie2 antibody 3H7H12G4 in a mouse model of primary open-angle glaucoma. Tamoxifen administration for inducible deletion of both angiopoietin-1 and -2 was performed at 8-weeks-old A1:A2^{iΔ/Δ} mice. The intraocular administration of 3H7H12G4 (one eye, 1 μl injection of 5 mg/ml solution) and Fc (contralateral eye, 1 μl injection of 5 mg/ml solution) was performed at 12 weeks old. Periodic measurement of intraocular pressure (IOP) was performed at 12, 13 and 14 weeks old. CD144⁺ SC area and intensities of Prox1 and Tie2 immunostaining in CD144⁺ SC were measured at 2 weeks after 3H7H12G4 administration. Scale bar, 100 μm. n=5 for each group. Values are mean±SD. *p<0.05 by Kruskal-Wallis test followed by Tukey's HSD test with ranks.

FIGS. 9A-9C shows suppression of CNV (Choroidal Neo-Vascularization) and vascular leakage by intravitreally injected 3H7H12G4 in laser-induced CNV model. The intravitreal administration of antibodies (1 μl injection of 5 mg/ml solution) was performed at 7 days after laser photocoagulation. CD31+CNV volumes were measured and leaky areas around CNV were calculated as the total measured hyper-fluorescent areas in FA images divided by the total measured CNV areas in ICGA images at 6 and/or 14 days after laser photocoagulation. n=11 for each group. Values are mean±SD. *p<0.05, ***p<0.001 by one-way ANOVA followed by Student-Newman-Keuls post-test: ^{##}p<0.01, ^{###}p<0.001 by paired Student's t-test.

FIGS. 10A-10D is the result about co-localization of 3H7H12G4 and CD31 in endothelial cells of CNV area. The subcutaneous administration of 3H7H12G4 was performed at 1 day after laser photocoagulation. The co-localization of 3H7H12G4 and CD31in endothelial cells of CNV was directly detected by anti-human IgG secondary antibody at 2, 4, and 8 days after laser photocoagulation.

FIGS. 11A-11C is the result about suppression by subcutaneously injected 3H7H12G4. The subcutaneous administration of 3H7H12G4 was performed at 1 day after laser photocoagulation. CD31⁺ CNV volumes were measured at 8 days after laser photocoagulation. Scale bar, 100 μm. n=10 for each group. Values are mean±SD. ***p<0.001 by unpaired Student's t-test.

### DETAILED DESCRIPTION AND PREFERRED EMBODIMENTS OF THE INVENTION

Unless defined otherwise, all technical and scientific terms used herein have the same meaning as those appreciated by those skilled in the field to which the present disclosure pertains.

The inventors of the present application confirmed Tie2 antibody as an increasing agent of Tie2 activity by binding to the Tie2 Ig3-FNIII(1-3) domain containing the sequence of SEQ ID NO:2.

Tie2 is a receptor protein that promotes the differentiation and stabilization of blood vessels, highly expressed in blood vessels, and if activated, the Tie2 receptor stabilizes cancer blood vessels and it becomes possible to gather surrounding support cells. By the antibodies or antigen-binding fragment thereof according to the invention, activated Tie2 in cancer vessels normalizes cancer vessels, eliminating the increased hypoxia within the tumor, supplying the sufficient oxygen by increasing blood flow into the tumor, and increasing the delivery of other anticancer drugs and the penetration of immune cells.

In this respect, the present invention relates to a Tie2 antibody or antigen-binding fragment thereof that binds to the Tie2 Ig3-FNIII(1-3) domain comprising the sequence of SEQ ID NO: 2.

As used herein, the term “antibody” means an antibody specifically binding to Tie2. In the scope of the present invention, in addition to a complete antibody specifically binding to Tie2, antigen-binding fragments of the antibody molecule are also included.

A complete antibody has the structure of two full-length light chains and two full-length heavy chains, and each light chain is connected to a heavy chain by a disulfide bond. The constant region of the heavy chain has gamma (γ), mu (μ), alpha (α), delta (δ) and epsilon (ε) types, with the subclasses of Gamma 1 (γ1), Gamma 2 (γ2), Gamma 3 (γ3), Gamma 4 (γ4), Alpha 1 (α1) and alpha 2 (α2). The constant region of the light chain has kappa (κ) and lambda (λ) types.

Antigen-binding fragments or antibody fragments of an antibody means a fragment which can bind to an antigen, and includes Fab, F(ab′), F(ab′)2 and Fv. Among antibody fragments, Fab bas one antigen-binding site with a structure of the variable regions of the light and heavy chain, the constant region of the light chain, and the first CHI of the heavy chain.

Fab′ differs from Fab in that it has a hinge region comprising one or more cysteine residues at the C-terminus of the CH1 domain. F(ab′)2 antibody is produced by disulfie bonds formation between Cysteine residues in the region of the hinge of Fab′. Fv is the smallest antibody fragment having only the variable region of the heavy chain and the variable region of the light chain. Double chain Fv (two-chain Fv) is formed by a non-covalent bond between the heavy chain variable region and the light chain variable region, and single-chain Fv (scFv) is generally formed through a peptide linker covalently between the variable region of the heavy chain and the variable region of the light chain, or is connected directly at the C-terminus by forming a dimer-like structure like a double-chain Fv. This fragment can be obtained by protein hydrolysis enzyme (e.g., you can get Fab by restriction digestion of whole antibody using papain, you can get F(ab′)2 fragment by cutting with pepsin), also made by genetic manipulation technology.

In one embodiment, the antibody according to the invention is the Fv form (e.g., scFv), or a complete antibody form. In addition, constant region of the heavy chain may be selected from any isotypes of gamma (γ), mu (μ), alpha (α), delta (δ), or epsilon (ε). For example, the constant region is gamma 1 (IgG1), gamma 3 (IgG3), or gamma 4 (IgG4). The light chain constant region can be of kappa or lambda type.

The term in the present invention, “heavy chain”, means the full-length heavy chain or fragments thereof comprising a variable region domain VH and three constant region domains CH1, CH2 and CH3, having an amino acid sequence with a sufficient variable region in order to provide antigen specificity. In addition, the term in the present invention. “light chain”, means the full-length light chain or fragments thereof comprising a variable region domain VL and a constant region domain CL, having an amino acid sequence with a sufficient variable region in order to provide antigen specificity.

The antibodies of the present invention are monoclonal antibodies, multispecific antibodies, human antibodies, humanized antibodies, chimeric antibodies, single chain Fvs (scFV), single chain antibodies, Fab fragments, F(ab′) fragments, Disulfide-binding Fvs (sdFV) and anti-idiotype (anti-Id) antibodies, or of the above antibodies epitope-binding fragments and the like are included, but are not limited thereto.

The monoclonal antibody is an antibody obtained from a population of substantially homogeneous antibodies, i.e. refers to the same individual antibodies in the population which may be present in trace amounts except for the possible natural mutation. Monoclonal antibody is highly specific, and it is induced against a single antigenic site. In contrast to the conventional (polyclonal) antibody which typically includes different antibodies instructed by different epitopes, each monoclonal antibody is instructed by a single determining factor.

“Epitope” means a protein determinant to which an antibody can specifically bind to. Epitopes are usually a group of chemically active surface molecules, e.g. it is composed of amino acids or sugar side chains, and generally has a specific charge characteristic as well as a specific three-dimensional structural characteristic. Stereoscopic epitope and non-stereoscopic epitope lose its bonds to the former in the presence of a denaturing solvent, but it does not lose bonds to the latter.

The Tie2 antibody or antigen-binding fragment thereof according to the present invention, when the epitope is identified through hydrogen/deuterium exchange, binds to 633 to 644 amino acids of Tie2 comprising the sequence of SEQ ID NO: 1 TLSDILPPQPEN and/or at amino acids 713 to 726 FAENNIGSSNPAFS.

The “humanized” form of non-human (e.g. murine) antibody is a chimeric antibody comprising one or more amino acid sequences (e.g. CDR sequence) from one or more non-human antibodies (donor or source antibodies) having a minimal sequence derived from non-human immunoglobulins. In most cases, the humanized antibody is a human immunoglobulin (recipient antibody) whose residues of hypervariable region are replaced by residues from hypervariable regions of non-human species (donor antibody), for example, mouse, rat, rabbit or non-human primate, possessing the desired specificity, affinity and ability. For humanization, one or more residues in the framework domain (FR) of the variable regions of the recipient human antibody can be replaced by the corresponding residues from a non-human species donor antibody. Through this, it helps to maintain a proper three-dimensional configuration of the grafted CDR(s), thereby improving affinity and antibody stability. Humanized antibodies can include a new additional residue that does not appear in the recipient antibody or donor antibody, e.g., to further refine the performance of antibody.

The “humanized antibody” as a molecule derived from human immunoglobulin means that the entire amino acid sequence constituting the antibody, including the complementary determining region and structural region, is composed of human immunoglobulin.

Any “chimeric” antibodies (immunoglobulins) as well as the fragment of the above-mentioned antibody, which exhibit the desired biological activity, are included where part of the heavy and/or light chain derived from a particular species, or identical or homologous to the corresponding sequence in the antibody belonging to the subclass, while the remaining chain(s) are derived from another species, or belonging to other antibody classes or identical to the corresponding sequence in the antibody belonging to the subclass.

According to a specific example of the present application, 11C4, 4A4, 3B2, 3E12 and 3H7 antibodies, which are mouse-derived Tie2 antibodies, were produced, and 3H7H11G4, 3H7H12G4, 3H7H21G4 or 3H7H22G4 antibodies, which were CDR-grafted and humanized from the mouse-derived 3H7 antibody as a source antibody, were produced.

“Antibody variable domain” as used herein refers to light and heavy chain parts of an antibody molecule comprising the amino acid sequences of complementarity determining regions (CDR; i.e., CDR1, CDR2, and CDR3) and framework regions (FR). VH refers to the variable domain of the heavy chain. VL refers to the variable domain of the light chain.

“Complementarity determining regions” (CDRs; i.e., CDR1, CDR2, and CDR3) refers to the amino acid residues of the variable domain of the antibody, which are necessary for antigen binding. Each variable domain typically has three CDR regions identified as CDR1, CDR2 and CDR3.

In one embodiment, the Tie2 antibody or antigen-binding fragment thereof may include a heavy chain variable region comprising heavy chain CDRs comprising the amino acid sequences of SEQ ID NOs: 3-5, a light chain variable region comprising light chain CDRs comprising the amino acid sequences of SEQ ID NOs: 6 to 8;
a heavy chain variable region comprising heavy chain CDRs comprising the amino acid sequences of SEQ ID NOs: 13-15, a light chain variable region comprising light chain CDRs comprising the amino acid sequences of SEQ ID NOs: 16 to 18;
a heavy chain variable region comprising heavy chain CDRs comprising the amino acid sequences of SEQ ID NOs: 23-25, a light chain variable region comprising light chain CDRs comprising the amino acid sequences of SEQ ID NOs: 26 to 28;
a heavy chain variable region comprising heavy chain CDRs comprising the amino acid sequences of SEQ ID NOs: 33-35, a light chain variable region comprising light chain CDRs comprising the amino acid sequences of SEQ ID NOs: 36 to 38; or
a heavy chain variable region comprising heavy chain CDRs comprising the amino acid sequences of SEQ ID NOs: 43-45, a light chain variable region comprising light chain CDRs comprising the amino acid sequences of SEQ ID NOs: 46 to 48.

“Framework region” (FR) is a variable domain residue other than a CDR residue. Each variable domain is typically, has 4 FRs identified as FR1, FR2, FR3 and FR4.

Tie2 antibodies are monovalent or bivalent, and contain single or double chains. Functionally, the binding affinity of the Tie2 antibody is in the range of 10⁻⁵ M to 10⁻¹² M.

For example, the binding affinity of the Tie2 antibody is 10⁻⁶ M to 10⁻¹² M, 10⁻⁷ M to 10⁻¹² M, 10⁻⁸ M to 10⁻¹² M, 10⁻⁹ M to 10⁻¹² M, 10⁻⁵ M to 10⁻¹¹ M, 10⁻⁶ M to 10⁻¹¹ M, 10⁻⁷ M to 10⁻¹¹ M, 10⁻⁸ M to 10⁻¹¹ M, 10⁻⁹ M to 10⁻¹¹ M, 10⁻¹⁰ M to 10⁻¹¹ M, 10⁻⁵ M to 10⁻¹⁰ M, 10⁻⁶ M to 10⁻¹⁰ M, 10⁻⁷ M to 10⁻¹⁰ M, 10⁻⁸ M to 10⁻¹⁰ M, 10⁻⁹ M to 10⁻¹⁰ M, 10⁻⁵ M to 10⁻⁹ M, 10⁻⁶ M to 10⁻⁹ M, 10⁻⁷ M to 10⁻⁹ M, 10⁻⁸ M to 10⁻⁹ M, 10⁻⁵ M to 10⁻⁸ M, 10⁻⁶ M to 10⁻⁸ M, 10⁻⁷ M to 10⁻⁸ M, 10⁻⁵ M to 10⁻⁷ M, 10⁻⁶ M to 10⁻⁷ M or 10⁻⁵ M to 10⁻⁶ M.

The Tie2 antibody or antigen-binding fragment thereof may include a heavy chain variable region comprising the amino acid sequence of SEQ ID NO: 9 and a light chain variable region comprising the amino acid sequence of SEQ ID NO: 11;
a heavy chain variable region comprising the amino acid sequence of SEQ ID NO: 19 and a light chain variable region comprising the amino acid sequence of SEQ ID NO: 21;
a heavy chain variable region comprising the amino acid sequence of SEQ ID NO: 29 and a light chain variable region comprising the amino acid sequence of SEQ ID NO: 31;
a heavy chain variable region comprising the amino acid sequence of SEQ ID NO: 39 and a light chain variable region comprising the amino acid sequence of SEQ ID NO: 41;
a heavy chain variable region comprising the amino acid sequence of SEQ ID NO: 49 and a light chain variable region comprising the amino acid sequence of SEQ ID NO: 51;
a heavy chain variable region comprising the amino acid sequence of SEQ ID NO: 53 and a light chain variable region comprising the amino acid sequence of SEQ ID NO: 54;
a heavy chain variable region comprising the amino acid sequence of SEQ ID NO: 57 and a light chain variable region comprising the amino acid sequence of SEQ ID NO: 58;
a heavy chain variable region comprising the amino acid sequence of SEQ ID NO: 61 and a light chain variable region comprising the amino acid sequence of SEQ ID NO: 62;
a heavy chain variable region comprising the amino acid sequence of SEQ ID NO: 19 and a light chain variable region comprising the amino acid sequence of SEQ ID NO: 21.

The antibody or antibody fragment of the present invention can include its biological equivalent within a range that can specifically recognize Tie2. For example, it can introduce a change to the amino acid sequence to improve further the binding affinity and/or its other biological properties of the antibody. Such modifications can include, for example, deletion, insertions and/or substitutions of amino acid sequence residues of the antibody. These amino acid variations are made based on the relative similarity of the amino acid substituents, such as hydrophobicity, hydrophilicity, charge, size, etc. of the amino acid side chains. By the analysis of the size, shape and type of amino acid side chain substituents, it is known that arginine, lysine and histidine are all positively charged residues; alanine, glycine and serine have similar sizes; Phenylalanine, tryptophan and tyrosine have a similar shape. Therefore, based on these considerations, we can say that arginine, lysine and histidine; alanine, glycine and serine; and phenylalanine, tryptophan and tyrosine are biologically functional equivalents.

Considering the above-described variation having biologically equivalent activity, it is interpreted that the amino acid sequence of the antibody of the invention or the nucleic acid molecule encoding the same antibody include the sequence in SEQ ID NO and any sequence exhibiting substantial identity. The substantial identity means at least 90% homology, most preferably at least 95% homology, 96% or more, 97% or more, 98% or more, 99% or more sequence homology, when the sequence described in the present invention and any other sequence are aligned as much as possible and analyzed by commonly used algorithm used in the art. The alignment method for sequence comparison is known in the art. NCBI Basic Local Alignment Search Tool (BLAST) is accessible from NBCI, etc., and it can be used in conjunction with a sequence analysis program such as blastp, blasm, blastx, tblastn and tblastx in the internet. BLSAT is accessible at www.ncbi.nlm.nih.gov/BLAST/.

The sequence homology comparison method using this program can be found at www.ncbi.nlm.nih.gov/BLAST/blast_help.html.

Based on this, the antibody or antigen-binding fragment thereof of the present invention may have 90%, 91%, 92%, 93%, 94%, 95%, 96%, 97%, 98%, 99%, or more homology, when compared to the specified or all sequence described in the specification. This homology can be determined by sequence comparison and/or alignment by known methods in the art. For example, sequence comparison algorithm (i.e. BLAST or BLAST 2.0), manual alignment, visual inspection can be used to determine the percent sequence homology of a nucleic acid or protein of the present invention.

In another aspect, the present invention relates to nucleic acids encoding the antibody or antigen-binding fragment thereof.

The nucleic acid may comprise the sequence of SEQ ID NO: 10, 12, 20, 22, 30, 32, 40, 42, 50, 52, 55, 56, 59, 60, 63, 64, 67, or 68.

The antibody or the antigen-binding fragment thereof can be produced recombinantly by isolating the nucleic acid encoding the antibody or antigen-binding fragment thereof of the present invention. Further cloning (DNA amplification) by isolating nucleic acids, and inserting it into a replicable vector may be done or further expression may be made. Based on this, the present invention relates to the vector containing the nucleic acid in another aspect.

“Nucleic acid” is meant to encompass DNA (gDNA and eDNA) and RNA molecules inclusively, and nucleotide, the basic structural unit of nucleic acid, includes the nucleotide in nature, as well as the analogue with modified sugar or base moieties. The sequence of the nucleic acid encoding the heavy and light chain variable regions of the present invention can be modified. The modifications include addition, deletion, or non-conservative or conservative substitution of nucleotides.

The DNA encoding the antibody is easily separated or synthesized using a conventional process (for example, by using oligonucleotide probes capable of specifically binding to the DNA encoding the heavy and light chains). Many vectors are available. Vector components generally includes one or more of the following, but is not limited to: signal sequence, origin of replication, one or more marker genes, enhancer element, promoter, and transcription termination sequence.

As used herein, the term “vector”, as means for expression a gene of interest in a host cell, includes plasmid vectors, cozmid vector, bacteriophage vector, adenovirus vectors, retroviral vectors and viral vectors such as adeno-associated virus vectors. The nucleic acid encoding the antibody in the vector is operatively linked to a promoter.

“Operatively linked” means the functional linkage between a nucleic acid expression control sequence (for example, promoter, signal sequence, or array of transcriptional regulator binding sites) and different nucleic acid sequences, Thereby, the control sequence controls transcription and/or translation of the other nucleic acid sequence.

In the case of prokaryotic cell as a host, a strong promotor which can process the transcription (for example, tac promoter, lac promoter, lacUV5 promoter, lpp promoter, pLλ promoter, pRλ promoter, rac5 promoter, amp promoter, recA promoter, SP6 Promoter, trp promoter and T7 promoter, etc.), ribosome binding site for initiation of translation and transcription/translation termination sequences are generally included. In addition, for example, in the case of eukaryotic cell as a host, a promoter derived from the genome of mammalian cells (example: metallothionine promoter, β-actin promoter, human hemoglobin promoter and human muscle creatine promoter) or a promotor derived from mammalian viruses (example: adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, Moloney Virus promotor, Epstein Barr Virus (EBV) promoter, and Rous sarcoma virus (RSV) promoter) can be used, and a polyadenylation sequence can be included generally as a transcription termination sequence.

In some cases, the vector may be fused with other sequences to facilitate the purification of the antibody expressed. The sequence to be fused is, for example, glutathione S-transferase (Pharmacia, USA), maltose binding protein (NEB, USA), FLAG (IBI, USA) and 6x His (hexahistidine; Qiagen, USA).

The vector contains an antibiotic resistance gene commonly used in the art as a selection marker, for example a resistant gene to ampicillin, gentamicin, carbenicillin, chloramphenicol, streptomycin, kanamycin, geneticin, neomycin and tetracycline.

In another aspect, the present invention relates to a cell transformed with the above-mentioned vector. Cells used to produce the antibodies of the present invention may be prokaryote, yeast and a higher eukaryotic cell, but not limited thereto.

Prokaryotic host cells such as Escherichia coli, Bacillus strains such as Bacillus subtilis and Bacillus thuringiensis, Streptomyces, Pseudomonas (e.g. Pseudomonas putida), Proteus mirabilis and Staphylococcus (for example, Staphylococcus carnosus), can be used.

However, the interest in animal cells is the greatest, the examples of useful host cell lines are COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but not limited thereto.

In another aspect the present invention relates to, (a) the step for culture of the cell; and (b) the method of manufacturing the antibody and antigen-binding fragment thereof, including recovery step for the antibody or antigen-binding fragment thereof from the cultured cells.

The cells can be cultured in various media. Any commercial culture media can be used without limitation. All other essential supplements known to those skilled in the art may be included in an appropriate concentration. Culture conditions, such as temperature, pH, etc., and selected host cells have already been used, and this will be obvious to those skilled in the art.

Recovery of the antibody or antigen-binding fragment thereof can be made through removing impurities for example using centrifugation or ultrafiltration, and for example using affinity chromatography, etc. Additional extra purification technology such as anion or cation exchange chromatography, hydrophobic interaction chromatography, hydroxyapatite chromatography, and the like can be used.

In another aspect, the present invention relates to the composition containing an active ingredient of the antibody or antigen-binding fragment thereof for preventing or treating angiogenic diseases.

The angiogenesis means the formation or growth of new blood vessels from previously existing blood vessels, “Angiogenesis-related disease” means the occurrence of angiogenesis or a disease associated with progression. If it can be treated with the antibody, the disease may be included in the range of angiogenesis-related diseases without limitation. The examples of the angiogenesis-related diseases are selected from the group composing cancer, metastasis, diabetic retinopathy, retinopathy of prematurity, corneal graft rejection, macular degeneration, neovascular glaucoma, systemic erythrosis, proliferative retinopathy, psoriasis, hemophilic arthritis, capillary formation in atherosclerotic plaques, keloid, wound granulation, vascular adhesion, rheumatoid arthritis, degenerative osteoarthritis, autoimmune diseases, Crohn's disease, restenosis, atherosclerosis, Intestinal adhesions, cat scratch disease, ulcers, liver cirrhosis, nephritis, diabetic nephropathy, diabetes mellitus, inflammatory diseases and neurodegenerative disease, but not limited thereto. In addition, the cancer is selected from the group composing esophageal cancer, stomach cancer, large intestine cancer. rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid blood cancer, but not limited thereto.

The term “prevention” as used herein denotes any action to inhibit or delay the onset of the disease of interest by administering the antibody or composition of the present invention. The term “treatment or therapy” indicates any action that improves or gets better the symptoms of the disease of interest by administering the antibody or composition of the present invention.

The composition comprising the antibody of the present invention is preferably a pharmaceutical composition, and can include a suitable vehicle, excipient or diluent typically used in the field.

Pharmaceutical compositions having a pharmaceutically acceptable vehicle can be various oral or parenteral dosage form such as tablets, pills, powders, granules, capsules, suspensions, oral solutions, emulsions, syrups, sterile aqueous solutions, non-aqueous solutions, suspensions, lyophilizates, and suppository. In relation to the pharmaceutical composition of the present invention can be a diluent or excipient which can be formulated in combination, such as fillers, thickeners, binders, wetting agents, disintegrants, surfactants, etc. Solid preparations for oral administration may be in the form of tablets, pills, powders, granules, capsules, and the like. In connection with the solidarity the compound of the present invention can be formulated by combining one or more excipients such as starch, calcium carbonate, sucrose, lactose, or gelatin. Simple excipient and lubricating agents such as magnesium stearate, talc, and the like may additionally be used.

The liquid preparation for oral administration may be a suspension, an oral solution, an emulsion, a syrup, or the like. Excipients such as water or simple diluents like wet paraffin, a variety of wetting agents, sweeteners, aromatics, preservatives, and etc. can be included in a liquid formulation. In addition, the pharmaceutical compositions in the present invention may be in parenteral dosage form such as sterile aqueous solution, non-aqueous solvent, suspension, emulsion, lyophilisate, suppository, etc. Injectable propylene glycol, polyethylene glycol, vegetable oils such as olive oil and esters such as ethyl oleate may be suitable for insoluble solvent and suspension. The basic substance of the suppository includes Witepsol, macrogol, Tween 61, cacao butter, laurin butter and glycerogelatin.

The composition of the present invention is administered in a pharmaceutically effective amount. Terms used here “A pharmaceutically effective amount” refers to the enough amount of the pharmaceutical composition for disease treatment, with an appropriate benefit/risk ratio which can be applicable for all medical treatments. The effective amount can be different depending on various factors including parameters like the severity of the disease, the patient's age and sex, type of disease, drug activity, drug sensitivity, administration time, route of administration, secretion rate, duration of treatment, co-administration of drugs and other known in the art. The composition in the present invention can be administered by alone or combination with other treatment. In this case, it can be administered sequentially or simultaneously with conventional therapy. Also, the above composition can be administered in single doses or divided into multiple doses. When fully considering these factors, it is important to administer the minimum amount sufficient to obtain maximum effect without side effects and this dosage can be easily determined by an expert. The dosage of the pharmaceutical composition of the present invention is not particularly limited, but it varies depending on various factors, including patient's health status and weight, disease severity, drug type, administration route and administration time.

The composition may be administered into mammals, including rats, mice, livestock, humans, etc., by one time or multiple times a day, via typically accepted routes, for example, orally, rectal, intravenously, subcutaneously, Intrauterinely or intracerebrovascularly.

The present invention in other perspective refers to the prevention or treatment methods for angiogenic diseases, and anti-angiogenic methods, including the steps for administering into an individual in need of the antibody or the above composition.

The method of the present invention includes the procedures for administering a pharmaceutical composition of pharmaceutically effective dose for individuals in need of inhibition of angiogenesis. The object is a mammalian such as dog, cow, horse, rabbit, mouse, rat, chicken, and human, but it is not limited thereto. The pharmaceutical composition can be administered via a suitable way including parenterally, subcutaneously, intraperitoneally, intrapulmonarily or intranasally, and if necessary, intralesionally for local treatment. The preferred dosage of the pharmaceutical composition of the present invention is changing depending on various factors including the health status and weight of the individual, the severity of the disease, the type of drug, the route and time of administration, and it can be easily determined by those skilled in the art.

In other aspect the present invention refers to the methods of cancer prevention or treatment, including administering procedures of the composition or the antibody to an individual in need of the antibody and the composition or the pharmaceutical composition for cancer prevention or treatment including the antibody.

Cancer is not limited as long as it is treatable with the antibodies of the present invention. In detail the antibody of the present invention can prevent the occurrence or progression of cancer by inhibiting angiogenesis. Examples of the caner include esophageal cancer, stomach cancer, large intestine cancer, rectal cancer, oral cancer, pharynx cancer, larynx cancer, lung cancer, colon cancer, breast cancer, uterine cervical cancer, endometrial cancer, ovarian cancer, prostate cancer, testis cancer, bladder cancer, renal cancer, liver cancer, pancreatic cancer, bone cancer, connective tissue cancer, skin cancer, brain cancer, thyroid cancer, leukemia, Hodgkin's lymphoma, lymphoma, and multiple myeloid cancer blood cancer, but is not limited thereto.

In addition, the antibodies of the present invention can be used in combination with other antibodies or biologically active agents or a material for various purposes. The present invention in this respect refers to a composition for combined administration with other therapeutic agents for angiogenic diseases, including the antibody or the antigen-binding fragments thereof.

The other therapeutic agents for angiogenic diseases include anti-angiogenic drugs, anti-inflammatory drugs and/or anticancer drugs. Through this, we can overcome each other's resistance and improve efficacy.

In the composition according to the present invention, when administered in combination with other therapeutic agents for angiogenic diseases, the Tie2 antibody and other therapeutic agents for angiogenic diseases may be administered sequentially or simultaneously. For example, after administering anti-angiogenic drugs, anti-inflammatory drugs and/or anticancer drug into the target individual, the composition having an antibody to Tie2 or an antigen-binding fragment thereof as an active ingredient can be administered, or after the above composition is administered, anti-angiogenic drugs, anti-inflammatory drugs and/or anticancer drug can be administered. Depending on the case, the above composition and the anti-angiogenic drugs, anti-inflammatory drugs and/or anticancer drug can be administered simultaneously to the target individual.

### EXAMPLE

Hereafter, the present invention will be described in detail by examples. The following examples are intended merely to illustrate the invention and are not constructed to restrict the invention.

### Example 1. Preparation of Mouse Monoclonal Anti-Tie2 Antibody

1.1 Mouse Immunization with Human Tie2

As an immunogen, the Ig3-FNIII (1-3) domains of human Tie2 (hTie2-Ig3-FNIII (1-3), SEQ ID NO:2) was cloned into a vector containing CMV promotor and transiently expressed by transfecting into HEK293F cell line. After 5 days of incubation, the expressed recombinant hTie2-Ig3-FNIII (1-3) protein was purified by affinity column using Protein A. Five-week-old BALB/c mice were immunized with purified hTie2-Ig3-FNIII (1-3) (100 ug/injection) mixed with an adjuvant twice weekly for 6 weeks. Anti-Tie2 antibody titers in the sera of immunized mice were examined by human Tie2 (hTie2) ELISA kit (R&D Systems®). When the antibody titer (1:5,000 dilution) suitably increased (OD>1.0), the spleens were extracted from the immunized mice, and B lymphocytes were isolated therefrom and fused with cultured myeloma cells (SP2/0). The fused cells were cultured in a HAT medium containing hypoxanthine, aminopterin and thymidine, and hybridoma cells comprised only of a fusion of myeloma cells and B lymphocytes were selected therefrom and cultured. Survived hybridoma cells were seeded in 96-well plates and the culture supernatants were tested by hTie2 ELISA. Hybridoma pools showing a positive signal were selected for clonal selection through limiting dilution. Finally, 37 monoclonal hybridoma lines were established. Among them, several Tie2-binding antibodies showed Tie2-activating activity. Candidate antibodies were selected based on Tie2 activating level and high affinity against human Tie2, later processed for humanization.

TABLE 1
Human Tie2 full-length (hTie2) and Ig3-FNIII(1-3) sequences
Human Tie2 full-length (SEQ ID NO: 1)

| | |
|---|---|
| MDSLASLVLCGVSLLLSGTVEGAMDLILINSLPLVSDAETSLTCIASGWR | 50 |
| PHEPITIGRDFEALMNQHQDPLEVTQDVTREWAKKVVWKREKASKINGAY | 100 |
| FCEGRVRGEAIRIRTMKMRQQASFLPATLTMTVDKGDNVNISFKKVLIKE | 150 |
| EDAVIYKNGSFIHSVPRHEVPDILEVHLPHAQPQDAGVYSARYIGGNLFT | 200 |
| SAFTRLIVRRCEAQKWGPECNHLCTACMNNGVCHEDTGECICPPGFMGRT | 250 |
| CEKACELHTFGRTCKERCSGQEGCKSYVFCLPDPYGCSCATGWKGLQCNE | 300 |
| ACHPGFYGPDCKLRCSCNNGEMCDRFQGCLCSPGWQGLQCEREGIQRMTP | 350 |
| KIVDLPDHIEVNSGKFNPICKASGWPLPTNEEMTLVKPDGTVLHPKDFNH | 400 |
| TDHFSVAIFTIHRILPPDSGVWVCSVNTVAGMVEKPFNISVKVLPKPLNA | 450 |
| PNVIDTGHNFAVINISSEPYFGDGPIKSKKLLYKPVNHYEAWQHIQVTNE | 500 |
| IVTLNYLEPRTEYELCVQLVRRGEGGEGHPGPVRRFTTASIGLPPPRGLN | 550 |
| LLPKSQTTLNLTWQPIFPSSEDDFYVEVERRSVQKSDQQNIKVPGNLTSV | 600 |
| LLNNLHPREQYVVRARVNTKAQGEWSEDLTAWTLSDILPPQPENIKISNI | 650 |
| THSSAVISWTILDGYSISSITIRYKVQGKNEDQHVDVKIKNATITQYQLK | 700 |
| GLEPETAYQVDIFAENNIGSSNPAFSHELVTLPESQAPADLGGGKMLLIA | 750 |
| ILGSAGMTCLTVLLAFLIILQLKRANVQRRMAQAFQNVREEPAVQFNSGT | 800 |
| LALNRKVKNNPDPTIYPVLDWNDIKFQDVIGEGNFGQVLKARIKKDGLRM | 850 |
| DAAIKRMKEYASKDDHRDFAGELEVLCKLGHHPNIINLLGACEHRGYLYL | 900 |
| AIEYAPHGNLLDFLRKSRVLETDPAFAIANSTASTLSSQQLLHFAADVAR | 950 |
| GMDYLSQKQFIHRDLAARNILVGENYVAKIADFGLSRGQEVYVKKTMGRL | 1000 |
| PVRWMAIESLNYSVYTTNSDVWSYGVLLWEIVSLGGTPYCGMTCAELYEK | 1050 |
| LPQGYRLEKPLNCDDEVYDLMRQCWREKPYERPSFAQILVSLNRMLEERK | 1100 |
| TYVNTTLYEKFTYAGIDCSAEEAA | 1124 |
| Human Tie2 Ig3-FNIII(1-3) (SEQ ID NO: 2) | |
| TPKIVDLPDHIEVNSGKFNPICKASGWPLPTNEEMTLVKPDGTVLHPKDF | 50 |
| NHTDHFSVAIFTIHRILPPDSGVWVCSVNTVAGMVEKPFNISVKVLPKPL | 100 |
| NAPNVIDTGHNFAVINISSEPYFGDGPIKSKKLLYKPVNHYEAWQHIQVT | 150 |
| NEIVTLNYLEPRTEYELCVQLVRRGEGGEGHPGPVRRFTTASIGLPPPRG | 200 |
| LNLLPKSQTTLNLTWQPIFPSSEDDFYVEVERRSVQKSDQQNIKVPGNLT | 250 |
| SVLLNNLHPREQYVVRARVNTKAQGEWSEDLTAWTLSDILPPQPENIKIS | 300 |
| NITHSSAVISWTILDGYSISSITIRYKVQGKNEDQHVDVKIKNATITQYQ | 350 |
| LKGLEPETAYQVDIFAENNIGSSNPAFSHELVTLPESQAP | 390 |

### 1.2 Production and Purification of Mouse Monoclonal Anti-Tie2 Antibodies

In order to produce the anti-Tie2 antibodies selected based on the ELISA positive signals, hybridoma cells were cultured in 10% FBS-containing DMEM (Dulbecco's Modified Eagle's Medium) in a T75 (75 cm² area) flask. When the confluency of the cells reached about 90%, the cells were washed with PBS, incubated with 50 ml of serum-free medium (SFM, Gibco™) and cultured at 37° C. for 3 days. Then, the culture medium in which the antibody was secreted from each monoclonal hybridoma was collected, centrifuged to remove the cells, and the culture supernatant was collected and filtered. The antibody was then purified using an AKTA purification device (GE Healthcare) equipped with a Protein G affinity column (GE Healthcare). The purified antibody was concentrated by substituting the supernatant with PBS using a centrifugal filter unit (Amicon®).

### 1.3. Identification and Screening of Agonistic Tie2 Antibodies

To investigate whether the mouse anti-Tie2 antibodies induce the downstream signaling of the Tie2 receptor in endothelial cells, HUVECs (Lonza™) were treated with an anti-Tie2 antibodies, and then the level of Akt phosphorylation, the main downstream signaling protein of Tie2 receptor, was analyzed by immunoblotting. As a positive control, COMP-Ang1 (CA1) was treated into the cells.

Specifically, HUVECs (1×10⁵ cells/ml) were cultured in EGM-2 medium (Lonza™) at 37° C. in a 60 mm culture dish. Cells at 90% confluency were incubated with serum-free EBM-2 medium for 6 hr for serum starvation. The serum-starved HUVECs were treated with an anti-Tie2 antibodies and further incubated for 30 min. The cells were washed with cold PBS, treated with lysis buffer, and lysed at 4° C. for 20 min. Then, the cell lysates were prepared by centrifugation at 13000 rpm for 15 min. By adding 5× SDS sample buffer, cell lysate were prepared and the cell lysates were subjected to SDS PAGE and proteins were transferred to a nitrocellulose membrane (GE).

To investigate Akt phosphorylation, the blot was blocked with 5% skim milk-containing TBS-T at room temperature (RT) for 1 hr, and incubated with anti-phospho-Akt antibody (S473) at 4° C. for about 8 hr. The signals of phospho-Akt were visualized by an enhanced chemiluminescence (ECL). Then, the membrane was incubated in a stripping buffer (Thermo Scientific) for 15 min, and then reprobed with an anti-Akt antibody to determine the amount of total Akt.

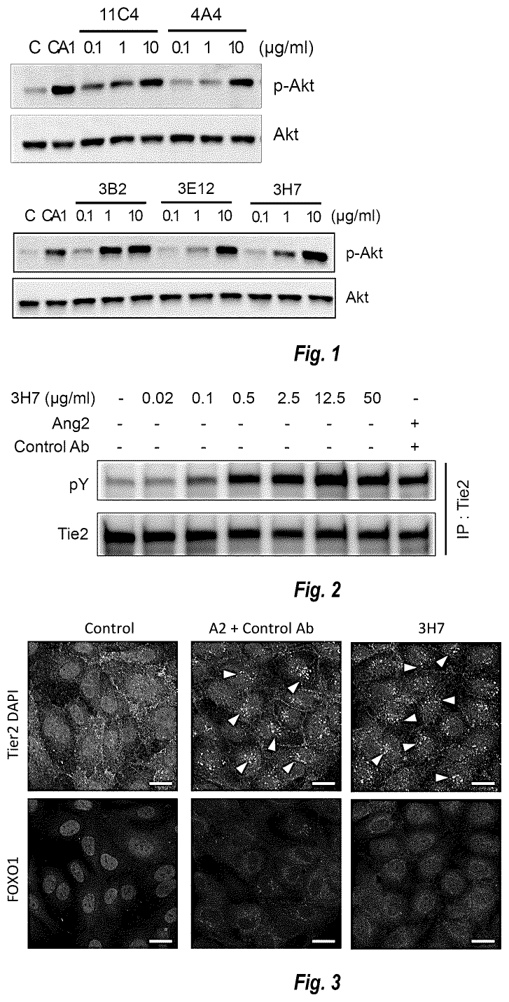

Akt phosphorylation at S473 was strongly induced in several groups treated with an anti-Tie2 antibody such as 11C4, 4A4, 3B2, 3E12 and 3H7 (FIG. 1).

1.4. Affinity Measurement of Anti-Tie2 Antibodies Against hTie2 by Octet® Analysis

The affinity of mouse monoclonal antibody against hTie2 was measured using Octet® system (ForteBio) where Black 96-well plates (96 well F-type black plates, Greiner) were used. The biosensor used for affinity measurements was hydrated for 10 min before measurement with AR2G tip (ForteBio Octet®). After the hydration, hTie2 was diluted in 10 mM sodium acetate, pH 6.0 buffer at a concentration of 10 μg/ml, fixed on AR2G biosensor, and blocked with 1M ethanolamine. The mouse monoclonal anti-Tie2 antibodies were diluted to 50, 25, 12.5, 6.25, 3.125, and 0 nM with 1× kinetic buffer, and subjected to association for 300 seconds and dissociation for 900 seconds. For affinity measurement (K_{D}), the association rate (K-on) and dissociation rate (K-off) were analyzed by binding curve (global) and fitted to 1:1 binding model using Octet® data analysis v9.0.0.10 program. The affinities of mouse anti-Tie2 antibodies are shown in Table 2.

TABLE 2
Affinities to hTie2 of mouse anti-Tie2 antibodies

| Antibody | Kon (1/Ms) | Koff (1/s) | K_{D} (M) |
|---|---|---|---|
| 11C4 | 1.52E+06 | 3.27E−04 | 2.15E−10 |
| 4A4 | 5.25E+05 | 5.53E−05 | 1.05E−10 |
| 3B2 | 4.44E+05 | <1.0E−07 | <1.0E−12 |
| 3E12 | 3.63E+05 | 9.12E−05 | 2.51E−10 |
| 3H7 | 3.84E+05 | 2.13E−05 | 5.56E−11 |

### 1.5. Tie2 Phosphorylation Induced by Mouse Tie2 Antibody 3H7

The anti-Tie2 antibodies developed in this invention are shown to bind and induce Tie2 clustering, ultimately triggering Tie2 activation. Experiments were conducted to analyze the effect of anti-Tie2 antibody on Tie2 phosphorylation using HUVECs.

Specifically, HUVECs (Lonza™) were cultured in EGM-2 (Lonza™) at 37° C. and 5% CO₂ concentration in a 100 mm culture dish. At 80-90% confluency, the cells were changed to EBM-2 (Lonza™) medium for 6 hr for serum starvation. Anti-Tie2 antibody at various concentrations (0.02 μg/ml to 50 μg/ml) were treated on the cultured cells and further incubated for 30 min. The cells were washed twice with cold PBS, lysed in 1000 μl of lysis buffer (10 mM Tris-Cl pH 7.4, 150 mM NaCl, 5 mM EDTA, 10% glycerol, 1% Triton™ X-100, protease inhibitor, phosphatase inhibitor) and then incubated at 4° C. for 60 min. Cell extracts were prepared and centrifuged at 12,000 rpm for 10 min. Protein concentration in the supernatant was quantitated by BCA assay.

For Tie2 immunoprecipitation, 1 μg of Tie2 antibody (R&D Systems®, AF313) was added into 0.5 mg of lysates and incubated overnight at 4° C. with shaking. Then, Dynabeads™ Protein G (Life Technologies) was added to react for 2 hrs. The beads were immobilized on one side of the tube using a magnet, washed three times with lysis buffer, and then incubated at 70° C. for 10 min with 2× SDS sample buffer containing reducing agent. The beads were removed from the sample and electrophoresed on a 4-15% SDS protein gel (Bio-Rad Laboratories) and then transferred to a 0.45 μm PVDF membrane.

The membrane was blocked with TBS-T mixed with 5% (v/v) BSA at room temperature for 1 hr and incubated with anti-phospho tyrosine antibody (4G10, Millipore®) at 4° C. for 8 hr, followed by the incubation of HRP-conjugated anti-mouse antibody and subsequent Western blotting analysis. To measure the amount of immunoprecipitated Tie2, the membrane was reacted in a stripping buffer (Thermo Scientific) for 15 min, then blocked again and reprobed with anti-Tie2 antibody (R&D Systems®, AF313). As shown in FIG. 2, when the anti-Tie2 antibody 3H7 was added to the HUVEC cells, the phosphorylation of Tie2 was strongly induced in a dose-dependent manner. These data indicate that the anti-Tie2 antibody 3H7 directly induce the activation of Tie2 receptor in human endothelial cells.

### 1.6. Clustered-Tie2 Endocytosis and FOXO1 Translocation in HUVECs by Tie2 Antibody (3H7)

The effect of 3H7 on the Tie2 localization and FOXO1 translocation from nucleus to cytosol was examined in HUVECs by immunofluorescence. Specifically, HUVECs were seeded on 8 well slide chamber (Lab-Tek™ II) and maintained in EGM-2 medium for 2˜3 days. At 100% confluence, the cells were serum staved with EBM-2 medium for 4 hr and then treated with 1 μg/ml of anti-Tie2 antibody 3H7 for 30 min. Thereafter, the cells were fixed with 4% formaldehyde in PBS at room temperature (RT) for 10 min, permeabilized with 0.1% Triton™ X-100 in PBS, blocked with 1% BSA in PBS at RT for 60 min, and incubated with primary antibodies at RT for 1 hr. The primary antibodies for hTie2 and FOXO1 were used. The cells were then incubated with secondary antibodies (Invitrogen) in the dark at RT for 1 hr and mounted with Vectashield® mounting medium with DAPI (Vector Labs). Images were taken with a laser scanning confocal microscope (LSM880, Carl Zeiss).

As shown in FIG. 3, the treatment of 3H7 markedly induced Tie2 endocytosis just like Control Ang2 antibody, which was known to induce Tie2 clustering and activation (Han et al., 2016, Science Translation Medicine). Consistent with a previous report showing FOXO1 localization in the cytoplasm after phosphorylation (Zhang et al., JBC 2002, 277, 45276-45284) while it was located in the nucleus under the basal, serum-starved condition, FOXO1 became markedly disappeared in nucleus with the treatment of 3H7, compared to serum-starved control.

1.7. Inhibition of Vascular Permeability Induced by VEGF or TNF-a when Treated Anti-Tie2 Antibody 3H7 in HUVECs.

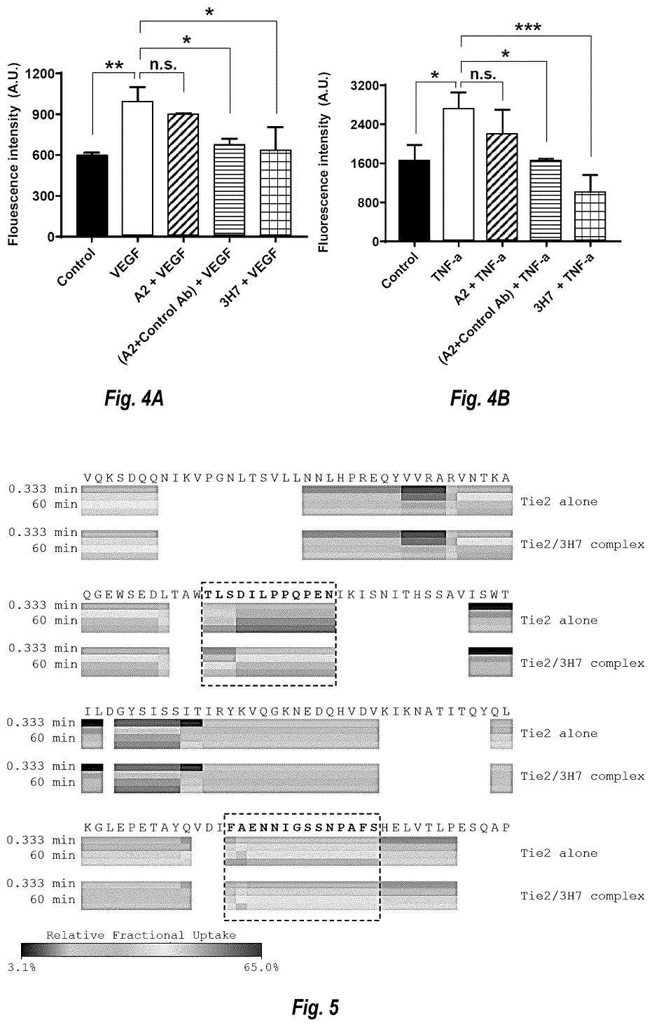

Vascular leakage assay was carried out in HUVECs using In Vitro Vascular Permeability Assay Kit (Millipore®) according to the manufacturer's instruction. HUVECs were seeded into the insert of the transwell plate and cultured for 3 days for 100% confluence. The HUVECs were pre-incubated with Ang2 (1 μg/ml), Ang2 (1 μg/ml) together with control antibody (1 μg/ml), or 3H7 antibody (1 μg/ml) alone for 30 min, and then VEGF (500 ng/ml) or TNF-a (100 ng/ml) was added, and the cells were incubated at 37° C. for 45 min or 22 hr, respectively. FITC-dextran was added to the upper chamber and incubated for 20 min. Passage of FITC-dextran though the HUVEC monolayer was measured by a fluorescence reader at excitation and emission wavelengths of 485 and 535 nm, respectively. As shown in FIG. 4, pre-treatment of anti-Tie2 antibody 3H7 significantly inhibited the vascular leakage induced by vascular-leakage promoting factor VEGF or TNF-a.

### Example 2. DNA Gene Sequence Analysis of Mouse Anti-Tie2 Antibodies

The DNA base sequences of the antibodies (derived from hybridoma cells) selected in Example 1.3 were analyzed. Specifically, hybridoma cells (2×10⁶ cells/ml) were cultured in 10% FBS-containing DMEM and then total RNA was obtained using RNeasy mini kit (Qiagen). Next, RNA concentration was measured, and cDNA was synthesized through reverse transcription (RT) reaction. To amplify the heavy and light chain variable region gene sequences, PCR was carried out using Mouse Ig-Primer set (Novagen®) under the following conditions using above cDNA as a template: 94° C. 5 min; [1 min at 94° C., 1 min at 50° C., 2 min at 72° C.]×35 cycles; 6 min at 72° C.; cooling to 4° C. The PCR product obtained from each reaction was cloned into a TA vector, and subjected to DNA sequencing, thereby obtaining the base sequences encoding the CDR, heavy-chain variable region and light-chain variable region of each antibody (Tables 3 to 12).

TABLE 3
CDR sequence of mouse anti-Tie2 antibody 3B2

| Antibody | CDR Sequence | | |
|---|---|---|---|
| 3B2 | Heavy Chain CDR Sequence | | |
| | CDRH1-KABAT | CDRH2-KABAT | CDRH3-KABAT |
| | SYWMN | MIHPSDSETRLNQKFKD | GNYFDC |
| | (SEQ ID NO: 3) | (SEQ ID NO: 4) | (SEQ ID NO: 5) |
| | Light Chain CDR Sequence | | |
| | CDRL1-KABAT | CDRL2-KABAT | CDRL3-KABAT |
| | RASQDIGISLN | ATSILDS | LQYASSPWT |
| | (SEQ ID NO: 6) | (SEQ ID NO: 7) | (SEQ ID NO: 8) |

TABLE 4
Variable region sequence of mouse anti-Tie2 antibody 3B2

| Antibody | Variable Region Sequence |
|---|---|
| 3B2 | Heavy Chain Variable Region Sequence |
| | QVQLQQPGAELVRPGASVKLSCKASGYSFTSYWMNWVKQRPGQGLEWIGMIHPS |
| | DSETRLNQKFKDKATLTVDKSSSTAYLQLSSPTSEDSAVYYCARGNYFDCWGQG |
| | TTLTVSS |
| | (SEQ ID NO: 9) |
| | CAGGTCCAACTGCAGCAGCCTGGGGCTGAGCTGGTGAGGCCTGGAGCTTCAGT |
| | GAAGCTGTCCTGCAAGGCTTCTGGCTACTCCTTCACCAGCTACTGGATGAACTG |
| | GGTGAAGCAGAGGCCTGGACAAGGCCTTGAGTGGATTGGCATGATTCATCCTTC |
| | CGATAGTGAAACTAGGTTAAATCAGAAGTTCAAGGACAAGGCCACATTGACTGT |
| | AGACAAATCCTCCAGCACAGCCTACTTGCAACTCAGCAGCCCGACATCTGAGGA |
| | CTCTGCGGTCTATTACTGTGCAAGGGGGAACTACTTTGACTGCTGGGGCCAAGG |
| | CACCACTCTCACAGTCTCCTCA |
| | (SEQ ID NO: 10) |
| | Light Chain Variable Region Sequence |
| | DIQMTQSPSSLSASLGERVSLTCRASQDIGISLNWLQQEPDGTIKRLIYATSILDSGV |
| | PKRFSGSRSGSDYSLTISSLESEDFVDYYCLQYASSPWTFGGGTKLEIK |
| | (SEQ ID NO: 11) |
| | GACATCCAGATGACCCAGTCTCCATCCTCCTTATCTGCCTCTCTGGGAGAAAGGG |
| | TCAGTCTCACTTGTCGGGCAAGTCAGGACATTGGTATTAGCTTAAACTGGCTTCA |
| | GCAGGAACCAGATGGAACTATTAAACGCCTGATCTACGCCACATCCATTTTAGAT |
| | TCTGGTGTCCCCAAAAGGTTCAGTGGCAGTAGGTCTGGGTCAGATTATTCTCTCA |
| | CCATCAGCAGCCTTGAGTCTGAAGATTTTGTAGACTATTACTGTCTACAATATGCT |
| | AGTTCTCCGTGGACGTTCGGTGGAGGCACCAAGCTGGAAATCAAA |
| | (SEQ ID NO: 12) |

TABLE 5
CDR sequence of mouse anti-Tie2 antibody 3E12

| Antibody | CDR Sequence | | |
|---|---|---|---|
| 3E12 | Heavy Chain CDR Sequence | | |
| | CDRH1-KABAT | CDRH2-KABAT | CDRH3-KABAT |
| | SYWMN | MIHPSDSETRLNQKFKD | GYYFGY |
| | (SEQ ID NO: 13) | (SEQ ID NO: 14) | (SEQ ID NO: 15) |
| | Light Chain CDR Sequence | | |
| | CDRL1-KABAT | CDRL2-KABAT | CDRL3-KABAT |
| | RASQDIGISLN | ATSNLDS | LQYASSPPT |
| | (SEQ ID NO: 16) | (SEQ ID NO: 17) | (SEQ ID NO: 18) |

TABLE 6
Variable region sequence of mouse anti-Tie2 antibody 3E12

| Antibody | Variable Region Sequence |
|---|---|
| 3E12 | Heavy Chain Variable Region Sequence |
| | QVQLQQPGADLVRPGASVKLSCKASGYSFTSYWMNWVKQRPGQGLEWIGMIHPS |
| | DSETRLNQKFKDKATLTVDKSSSTAYMQLSSPTSEDSAVYYCAKGYYFGYWGQ |
| | GTTLTVSS |
| | (SEQ ID NO: 19) |
| | CAGGTCCAACTGCAGCAGCCTGGGGCTGACCTGGTGAGGCCTGGAGCTTCAGT |
| | GAAGCTGTCCTGCAAGGCTTCTGGCTACTCCTTCACCAGCTACTGGATGAACTG |
| | GGTGAAGCAGAGGCCTGGACAAGGCCTTGAGTGGATTGGCATGATTCATCCTTC |
| | CGATAGTGAAACTAGGTTAAATCAGAAGTTCAAGGACAAGGCCACATTGACTGT |
| | AGACAAATCCTCCAGCACAGCCTACATGCAACTCAGCAGCCCGACATCTGAGGA |
| | CTCTGCGGTCTATTACTGTGCAAAGGGGTACTACTTTGGCTACTGGGGCCAAGGC |
| | ACCACTCTCACAGTCTCCTCA |
| | (SEQ ID NO: 20) |
| | Light Chain Variable Region Sequence |
| | DIQMTQSPSSLSASLGERVSLTCRASQDIGISLNWLQQEPDGTIKRLIYATSNLDSG |
| | VPKRFSGSRSGSDYSLTISSLESEDFVDYYCLQYASSPPTFGGGTKLEIK |
| | (SEQ ID NO: 21) |
| | GACATCCAGATGACCCAGTCTCCATCCTCCTTATCTGCCTCTCTGGGAGAAAGAG |
| | TCAGTCTCACTTGTCGGGCAAGTCAGGACATTGGTATTAGTTTAAACTGGCTTCA |
| | GCAGGAACCAGATGGAACTATTAAACGCCTGATCTACGCCACATCCAATTTAGAT |
| | TCTGGTGTCCCCAAAAGGTTCAGTGGCAGTAGGTCTGGGTCAGATTATTCTCTCA |
| | CCATCAGCAGCCTTGAGTCTGAAGATTTTGTAGACTATTACTGTCTACAATATGCT |
| | AGTTCTCCTCCGACGTTCGGTGGAGGCACCAAGCTGGAAATCAAA |
| | (SEQ ID NO: 22) |

TABLE 7
CDR sequence of mouse anti-Tie2 antibody 3H7

| Antibody | CDR Sequence | | |
|---|---|---|---|
| 3H7 | Heavy Chain CDR Sequence | | |
| | CDRH1-KABAT | CDRH2-KABAT | CDRH3-KABAT |
| | SYWMN | MIHPSDSETRLNQKFMD | GLYGNS |
| | (SEQ ID NO: 23) | (SEQ ID NO: 24) | (SEQ ID NO: 25) |
| | Light Chain CDR Sequence | | |
| | CDRL1-KABAT | CDRL2-KABAT | CDRL3-KABAT |
| | RASQDIGISLN | ATSSLDS | LQYASSPYT |
| | (SEQ ID NO: 26) | (SEQ ID NO: 27) | (SEQ ID NO: 28) |

TABLE 8
Variable region sequence of mouse anti-Tie2 antibody 3H7

| Antibody | Variable Region Sequence |
|---|---|
| 3H7 | Heavy Chain Variable Region Sequence |
| | QVQLQQPGAELVRPGASVKLSCKASGYSFTSYWMNWVKQRPGQGLEWIGMIHPS |
| | DSETRLNQKFMDKATLTVDKSSSTAYMQLSSPTSEDSAVYYCARGLYGNSWGQ |
| | GTLVTVSA |
| | (SEQ ID NO: 29) |
| | CAGGTCCAACTGCAGCAGCCTGGGGCTGAGCTGGTGAGGCCTGGAGCTTCAGT |
| | GAAGCTGTCCTGCAAGGCTTCTGGCTACTCCTTCACCAGCTACTGGATGAACTG |
| | GGTGAAGCAGAGGCCTGGACAAGGCCTTGAGTGGATTGGCATGATTCATCCTTC |
| | CGATAGTGAAACTAGGTTAAATCAGAAGTTCATGGACAAGGCCACATTGACTGT |
| | AGACAAATCCTCCAGCACAGCCTACATGCAGCTCAGCAGCCCGACATCTGAGGA |
| | CTCTGCGGTCTATTACTGTGCTCGTGGCCTCTATGGTAACTCTTGGGGCCAAGGG |
| | ACTCTGGTCACTGTCTCTGCA |
| | (SEQ ID NO: 30) |
| | Light Chain Variable Region Sequence |
| | DIQMTQSPSSLSASLGERVSLTCRASQDIGISLNWLQQEPDGTIKRLIYATSSLDSG |
| | VPKRFSGSRSGSDYSLTISSLESEDFVDYYCLQYASSPYTFGGGTKLEIK |
| | (SEQ ID NO: 31) |
| | GACATCCAGATGACCCAGTCTCCATCCTCCTTATCTGCCTCTCTGGGAGAAAGAG |
| | TCAGTCTCACTTGTCGGGCAAGTCAGGACATTGGTATTAGCTTAAACTGGCTTCA |
| | GCAGGAACCAGATGGAACTATTAAACGCCTGATCTACGCCACATCCAGTTTAGAT |
| | TCTGGTGTCCCCAAAAGGTTCAGTGGCAGTAGGTCTGGGTCAGATTATTCTCTCA |
| | CCATCAGCAGCCTTGAGTCTGAAGATTTTGTAGACTATTACTGTCTACAATATGCT |
| | AGTTCTCCGTACACGTTCGGAGGGGGGACCAAGCTGGAAATAAAA |
| | (SEQ ID NO: 32) |

TABLE 9
CDR sequence of mouse anti-Tie2 antibody 4A4

| Antibody | CDR Sequence | | |
|---|---|---|---|
| 4A4 | Heavy Chain CDR Sequence | | |
| | CDRH1-KABAT | CDRH2-KABAT | CDRH3-KABAT |
| | SYWMN | MIHPSDSETRLNQKFKD | GYYFDY |
| | (SEQ ID NO: 33) | (SEQ ID NO: 34) | (SEQ ID NO: 35) |
| | Light Chain CDR Sequence | | |
| | CDRL1-KABAT | CDRL2-KABAT | CDRL3-KABAT |
| | RASQDIGISLN | ATSSLDS | LQYVSSPWT |
| | (SEQ ID NO: 36) | (SEQ ID NO: 37) | (SEQ ID NO: 38) |

TABLE 10
Variable region sequence of mouse anti-Tie2 antibody 4A4

| Antibody | Variable Region Sequence |
|---|---|
| 4A4 | Heavy Chain Variable Region Sequence |
| | QVQLQQPGAELVRPGASVKLSCKASGYSFTSYWMNWVKQRPGQGLEWIGMIHPS |
| | DSETRLNQKFKDKATLTVDKSSSTAYMQLSSPTSEDSAVYYCARGYYFDYWGQG |
| | TTLTVSS |
| | (SEQ ID NO: 39) |
| | CAGGTCCAACTGCAGCAGCCTGGGGCTGAGCTGGTGAGGCCTGGAGCTTCAGT |
| | GAAGCTGTCCTGCAAGGCTTCTGGCTACTCCTTCACCAGCTACTGGATGAACTG |
| | GGTGAAGCAGAGGCCTGGACAAGGCCTTGAGTGGATTGGCATGATTCATCCTTC |
| | CGATAGTGAAACTAGGTTAAATCAGAAGTTCAAGGACAAGGCCACATTGACTGT |
| | AGACAAATCCTCCAGCACAGCCTACATGCAACTCAGCAGCCCGACATCTGAGGA |
| | CTCTGCGGTCTATTACTGTGCAAGAGGCTACTACTTTGACTACTGGGGCCAAGGC |
| | ACCACTCTCACAGTCTCCTCA |
| | (SEQ ID NO: 40) |
| | Light Chain Variable Region Sequence |
| | DIQMTQSPSSLSASLGERVSLTCRASQDIGISLNWLQQEPDGTIKRLIYATSSLDSG |
| | VPKRFTGSRSGSDYSLTISSLESEDFVDYYCLQVSSPWTFGGGTKLEIK |
| | (SEQ ID NO: 41) |
| | GACATCCAGATGACCCAGTCTCCATCCTCCTTATCTGCCTCTCTGGGAGAAAGAG |
| | TCAGTCTCACTTGTCGGGCAAGTCAGGACATTGGTATTAGCTTAAACTGGCTTCA |
| | GCAGGAACCAGATGGAACTATTAAACGCCTGATCTACGCCACATCCAGTTTAGAT |
| | TCTGGTGTCCCCAAGAGGTTCACTGGCAGTAGGTCTGGGTCAGATTATTCTCTCA |
| | CCATCAGCAGCCTTGAGTCTGAAGATTTTGTAGACTATTACTGTCTACAATATGTT |
| | AGTTCTCCGTGGACGTTCGGTGGAGGCACCAAGCTGGAAATCAAA |
| | (SEQ ID NO: 42) |

TABLE 11
CDR sequence of mouse anti-Tie2 antibody 11C4

| Antibody | CDR Sequence | | |
|---|---|---|---|
| 11C4 | Heavy Chain CDR Sequence | | |
| | CDRH1-KABAT | CDRH2-KABAT | CDRH3-KABAT |
| | SYWMN | MIHPSDSETRLNQKFKD | LTIYFDY |
| | (SEQ ID NO: 43) | (SEQ ID NO: 44) | (SEQ ID NO: 45) |
| | Light Chain CDR Sequence | | |
| | CDRL1-KABAT | CDRL2-KABAT | CDRL3-KABAT |
| | RASQDIGISLN | ATSSLDS | LQYASSPYT |
| | (SEQ ID NO: 46) | (SEQ ID NO: 47) | (SEQ ID NO: 48) |

TABLE 12
Variable region sequence of mouse anti-Tie2 antibody 11C4

| Antibody | Variable Region Sequence |
|---|---|
| 11C4 | Heavy Chain Variable Region Sequence |
| | QVQLQQPGADLVRPGASVTLSCKASGYSFTSYWMNWVKQRPGQGLEWIGMIHPS |
| | DSETRLNQKFKDKATLTVDKSSSTAYMQLRSPTSEDSAVYYCAGLTIYFDYWGQ |
| | GTTLTVSS |
| | (SEQ ID NO: 49) |
| | CAGGTCCAACTACAGCAGCCTGGGGCTGACCTGGTGAGGCCTGGAGCTTCAGT |
| | GACGCTGTCCTGCAAGGCTTCTGGCTACTCCTTCACCAGCTACTGGATGAACTG |
| | GGTGAAGCAGAGGCCTGGACAAGGCCTGGAGTGGATTGGCATGATTCATCCTTC |
| | CGATAGTGAAACTAGGTTAAATCAGAAGTTCAAGGACAAGGCCACATTGACTGT |
| | AGACAAATCCTCCAGCACAGCCTACATGCAACTCCGCAGCCCGACATCTGAGGA |
| | CTCTGCGGTCTATTACTGTGCAGGCCTAACTATTTACTTTGACTATTGGGGCCAAG |
| | GCACCACTCTCACAGTCTCCTCA |
| | (SEQ ID NO: 50) |
| | Light Chain Variable Region Sequence |
| | DIQMTQSPSSLSASLGERVSLTCRASQDIGISLNWLQQEPDGTIKRLIYATSSLDSG |
| | VPKRFSGSRSGSDYSLTISSLESEDFVDYYCLQYASSPYTFGGGTKLEIK |
| | (SEQ ID NO: 51) |
| | GACATCCAGATGACCCAGTCTCCATCCTCCTTATCTGCCTCTCTGGGAGAAAGAG |
| | TCAGTCTCACTTGTCGGGCAAGTCAGGACATTGGTATTAGCTTAAACTGGCTTCA |
| | GCAGGAACCAGATGGAACTATTAAACGCCTGATCTACGCCACATCCAGTTTAGAT |
| | TCTGGTGTCCCCAAAAGGTTCAGTGGCAGTAGGTCTGGGTCAGATTATTCTCTCA |
| | CCATCAGCAGCCTTGAGTCTGAAGATTTTGTAGACTATTACTGTCTACAATATGCT |
| | AGTTCTCCGTACACGTTCGGAGGGGGGACCAAGCTGGAAATAAAA |
| | (SEQ ID NO: 52) |

### Example 3. Epitope Mapping of Mouse Anti-Tie2 Antibody Against hTie2

The antigenic determinants (epitopes) of hTie2 recognized by mouse monoclonal antibody 3H7 was analyzed by HDX-MS (Hydrogen/deuterium exchange-mass spectrometry) technique. HDX-MS analysis methods are described in the following articles; Houde D, Engen JR (2013) Methods Mol. Biol. 988:269-89 and Houde et al., (2011) J. Pharm. Sci. 100 (6), 2071.

Recombinant hTie2-Ig3-FNIII(1-3) protein was used to analyze the binding-epitopes of antibody 3H7. Before deuterium labeling reaction, hTie2-Ig3-FNIII(1-3)/antibody mixture was incubated for more than 3 hrs to be maintained to the maximum binding (100%) under 15× diluted deuterium labeling buffer (K_{D}=25 nM). The prepared hTie2-Ig3-FNIII(1-3)/antibody complex was diluted 15 times with deuterium labeling buffer, labeled at various time, and then quenched with the same volume of quenching buffer. The labeling reaction time was 0 min (undeuterium), 0.33 min, 10 min, 60 min and 240 min. However, in undeuterium condition, the deuterium labeling buffer was replaced with equilibrium buffer and the reaction was immediately stopped with quenching buffer. For mass spectrometry, the deuterium labeled hTie2-Ig3-FNIII(1-3)/antibody sample was loaded on a pepsin column and peptide digestion was carried out. Mass spectrometry analysis showed that 82.6% coverage data is obtained from a total of 50 peptic peptides.

The deuterium uptake difference between hTie2-Ig3-FNIII(1-3) alone and hTie2-Ig3-FNIII(1-3)/antibody complex conditions was comparatively analyzed, and a region showing a distinct decrease in the deuterium uptake is either a peptide to which the antibody binds directly, or a structurally changed region. When the deuterium uptake difference between hTie2-Ig3-FNIII(1-3) alone and the hTie2-Ig3-FNIII(1-3)/antibody complex was 0.5-1 Da or more, it was considered significant, indicated as bold in Table 13.

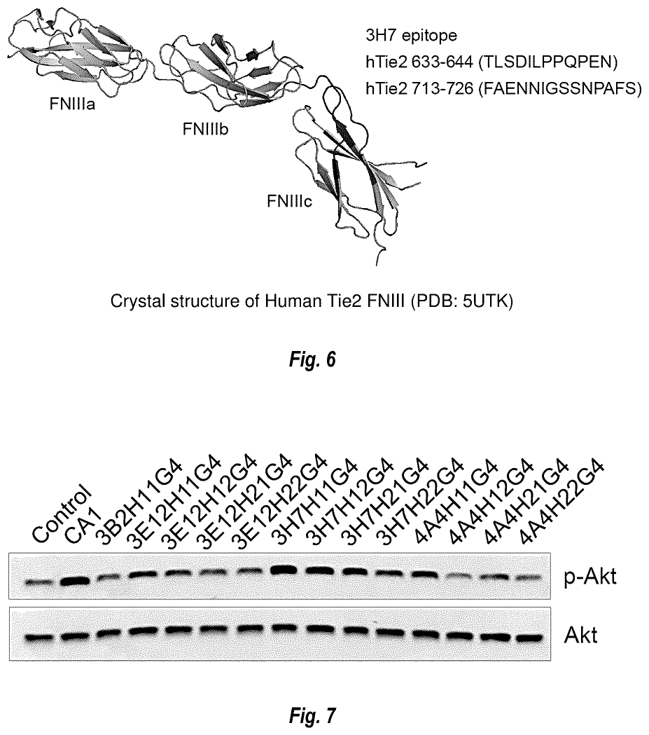

Heat map of deuterium uptake per residue at 0.333, 10, 60, and 240 min for hTie2 antigen of SEQ ID NO: 1 was generated in the absence or presence of anti-Tie2 antibody 3H7 using the data of H/D exchange-mass spectrometry (FIG. 5, residues 582 to 738 of SEQ ID NO: 1). Grey scale indicates the percentage of H/D exchange per residue between the antigen alone and antigen/mAb mixture at each individual time. The heat map analysis of the deuterium uptake difference indicated that the epitopes to which antibody 3H7 binds are residues 633 to 644 (SEQ ID NO: 1, TLSDILPPQPEN) and 713-726 (SEQ ID NO: 1, FAENNIGSSNPAFS) of hTie2 (Table 13). The epitope was shown on the FNIIIc of the 3D structure of hTie2-FNIII, which was generated using PyMol™ software (FIG. 6).

TABLE 13
Epitope mapping analysis for 3H7 binding to hTie2 by HDX-MS
3H7 binding to hTie2-Ig3-FNIII(1-3)

| Residues (SEQ ID NO: 2) | | | Exposure Time | Relative Uptake (Da) | | |
|---|---|---|---|---|---|---|
| Start | End | Sequence | (min) | hTie2 alone | hTie2 + 3H7 | Δ |
| 284 | 296 | WTLSDILPPQPEN | 0.00 | 0.00 | 0.00 | 0.00 |
| 284 | 296 | WTLSDILPPQPEN | 0.33 | 1.45 | 1.14 | 0.31 |
| 284 | 296 | WTLSDILPPQPEN | 10.00 | 3.09 | 2.62 | 0.47 |
| 284 | 296 | WTLSDILPPQPEN | 60.00 | 3.93 | 3.45 | 0.48 |
| 284 | 296 | WTLSDILPPQPEN | 240.00 | 4.50 | 3.99 | 0.51 |
| 287 | 296 | SDILPPQPEN | 0.00 | 0.00 | 0.00 | 0.00 |
| 287 | 296 | SDILPPQPEN | 0.33 | 1.31 | 0.99 | 0.33 |
| 287 | 296 | SDILPPQPEN | 10.00 | 2.79 | 2.32 | 0.48 |
| 287 | 296 | SDILPPQPEN | 60.00 | 3.20 | 2.70 | 0.51 |
| 287 | 296 | SDILPPQPEN | 240.00 | 3.49 | 2.92 | 0.57 |
| 308 | 314 | VISWTIL | 0.00 | 0.00 | 0.00 | 0.00 |
| 308 | 314 | VISWTIL | 0.33 | 0.07 | 0.07 | 0.00 |
| 308 | 314 | VISWTIL | 10.00 | 0.79 | 0.83 | -0.05 |
| 308 | 314 | VISWTIL | 60.00 | 1.48 | 1.56 | -0.08 |
| 308 | 314 | VISWTIL | 240.00 | 1.83 | 1.64 | 0.19 |
| 364 | 380 | IFAENNIGSSNPAFSHE | 0.00 | 0.00 | 0.00 | 0.00 |
| 364 | 380 | IFAENNIGSSNPAFSHE | 0.33 | 4.15 | 3.93 | 0.22 |
| 364 | 380 | IFAENNIGSSNPAFSHE | 10.00 | 5.31 | 4.62 | 0.70 |
| 364 | 380 | IFAENNIGSSNPAFSHE | 60.00 | 6.23 | 5.32 | 0.92 |
| 364 | 380 | IFAENNIGSSNPAFSHE | 240.00 | 7.15 | 6.15 | 1.00 |
| 365 | 380 | FAENNIGSSNPAFSHE | 0.00 | 0.00 | 0.00 | 0.00 |
| 365 | 380 | FAENNIGSSNPAFSHE | 0.33 | 4.40 | 4.21 | 0.19 |
| 365 | 380 | FAENNIGSSNPAFSHE | 10.00 | 5.42 | 5.05 | 0.37 |
| 365 | 380 | FAENNIGSSNPAFSHE | 60.00 | 6.08 | 5.60 | 0.47 |
| 365 | 380 | FAENNIGSSNPAFSHE | 240.00 | 6.81 | 6.11 | 0.70 |
| 366 | 380 | AENNIGSSNPAFSHE | 0.00 | 0.00 | 0.00 | 0.00 |
| 366 | 380 | AENNIGSSNPAFSHE | 0.33 | 4.04 | 3.69 | 0.35 |
| 366 | 380 | AENNIGSSNPAFSHE | 10.00 | 4.74 | 4.47 | 0.27 |
| 366 | 380 | AENNIGSSNPAFSHE | 60.00 | 5.35 | 5.08 | 0.27 |
| 366 | 380 | AENNIGSSNPAFSHE | 240.00 | 5.91 | 5.34 | 0.58 |
| 378 | 385 | SHELVTLP | 0.00 | 0.00 | 0.00 | 0.00 |
| 378 | 385 | SHELVTLP | 0.33 | 0.73 | 0.79 | -0.06 |
| 378 | 385 | SHELVTLP | 10.00 | 1.54 | 1.55 | -0.01 |
| 378 | 385 | SHELVTLP | 60.00 | 1.98 | 2.09 | -0.11 |
| 378 | 385 | SHELVTLP | 240.00 | 2.15 | 2.28 | -0.13 |

### Example 4. Humanization of Mouse Anti-Tie2 Antibody and Full-Length IgG Conversion

To eliminate the immunogenicity of mouse anti-Tie2 antibody 3H7 when administered into human, the antibody was humanized as follows.

### 4.1. Heavy Chain Humanization

The human antibody heavy chain variable gene, IGHV1-46-01, showed 66% homology to the heavy chain sequence of antibody 3H7. Based on these analyses, the 3 CDR regions of the 3H7 antibody were grafted into the human antibody heavy chain variable gene IGHV1-46-01. In this process, 2 humanized heavy chain antibody genes were designed (Table 14). The grafted CDRs are underlined in protein sequence. Back mutations to mouse sequence were introduced in heavy chain genes of humanized 3H7, indicated as bold in protein sequence of Table 14.

### 4.2 Light Chain Humanization

The human antibody light chain variable gene, IGKV1-17-01, showed 68% homology to the light chain sequence of antibody 3H7. Based on these analyses, the 3 CDR regions of 3H7 antibody were grafted into the human antibody light chain variable gene IGKV1-17-01. 2 humanized light chain antibody genes were designed in this process (Table 14). The grafted CDRs are underlined in protein sequence. Back mutations to mouse sequence were introduced in light chain genes of humanized 3H7, indicated as bold in protein sequence of Table 14.

### 4.3. Humanized Gene Synthesis and Cloning to Human Full-Length IgG Antibody

The humanized variable regions of antibodies in Table 14 were cloned into the heavy chain and the light chain vector of the human IgG4 isotype backbone vectors. DNA fragments of the humanized heavy chain variable region of the antibodies (VH) were synthesized (Bioneer, Inc.) as a sequence of ‘EcoRI-signal sequence-VH-NheI-CH-XhoI’. DNA fragments of the humanized light chain variable region of the antibodies (VL) were also synthesized as a sequence of ‘EcoRI-signal sequence-VL-BsiWI-CL-XhoI’. The DNA fragments encoding the heavy chain and light chain were cloned into pOptiVEC™ or pcDNA™3.3 vectors, respectively.

TABLE 14
Humanized anti-Tie2 antibodies originated from mouse 3H7 antibody

| Antibody | Antibody Sequence (VH) | Antibody Sequence (VL) |
|---|---|---|
| 3H7H11G4 | (Protein Sequence) | (Protein Sequence) |
| | QVQLVQSGAEVKKPGASVKVSCKA | DIQMTQSPSSLSASVGDRVTITCRAS |
| | SGYTFTSYWMNWVRQAPGQGLEW | QDIGISLNWYQQKPGKAPKRLIYAT |
| | MGMIHPSDSETRLNQKFMDRVTMT | SSLDSGVPSRFSGSGSGTEFTLTISSL |
| | RDTSTSTVYMELSSLRSEDTAVYYC | QPEDFATYYCLQYASSPYTFGQGTK |
| | ARGLYGNSWGQGTLVTVSS | VEIK |
| | (SEQ ID NO: 53) | (SEQ ID NO: 54) |
| | (Coding Nucleotide Sequence) | (Coding Nucleotide Sequence) |
| | CAGGTGCAGCTGGTCCAATCCGGG | GACATCCAGATGACCCAATCTCCCT |
| | GCTGAGGTGAAGAAGCCTGGAGC | CCTCCCTGAGCGCATCCGTGGGGG |
| | ATCAGTGAAAGTTTCATGCAAAGC | ATAGAGTGACCATAACCTGCCGGG |
| | TAGTGGTTACACCTTCACCAGCTAT | CCTCTCAGGACATCGGTATTTCTTT |
| | TGGATGAACTGGGTGCGGCAGGCC | GAATTGGTATCAGCAGAAGCCCGG |
| | CCCGGTCAGGGGCTTGAGTGGATG | GAAGGCCCCTAAACGCCTGATCTA |
| | GGCATGATCCACCCATCCGACTCTG | TGCTACTTCCAGTCTGGACAGCGG |
| | AGACTAGGCTGAACCAGAAGTTTA | GGTCCCGTCAAGGTTTTCAGGCAG |
| | TGGATAGAGTGACCATGACAAGAG | TGGATCAGGCACAGAGTTTACACT |
| | ATACGTCCACTTCTACTGTCTATATG | CACAATTTCGAGCCTGCAGCCTGA |
| | GAACTGAGCAGTCTGAGATCTGAA | AGATTTCGCCACTTATTACTGTCTT |
| | GACACAGCCGTTTACTACTGTGCT | CAATACGCTAGCTCTCCATACACGT |
| | CGCGGACTCTATGGCAATAGCTGG | TCGGCCAGGGAACCAAGGTTGAG |
| | GGCCAAGGAACATTGGTAACCGTC | ATTAAA |
| | TCTTCT | (SEQ ID NO: 56) |
| | (SEQ ID NO: 55) | |
| 3H7H12G4 | (Protein Sequence) | (Protein Sequence) |
| | QVQLVQSGAEVKKPGASVKVSCKA | DIQMTQSPSSLSASVGDRVTITCRAS |
| | SGYTFTSYWMNWVRQAPGQGLEW | QDIGISLNWLQQEPGKAPKRLIYAT |
| | MGMIHPSDSETRLNQKFMDRVTMT | SSLDSGVPKRFSGSGSGTEFTLTISSL |
| | RDTSTSTVYMELSSLRSEDTAVYYC | QPEDFATYYCLQYASSPYTFGQGTK |
| | ARGLYGNSWGQGTLVTVSS | VEIK |
| | (SEQ ID NO: 57) | (SEQ ID NO: 58) |
| | (Coding Nucleotide Sequence) | (Coding Nucleotide Sequence) |
| | CAGGTGCAGCTGGTCCAATCCGGG | GACATCCAGATGACTCAGTCCCCC |
| | GCTGAGGTGAAGAAGCCTGGAGC | TCGAGCCTCTCAGCTTCTGTTGGA |
| | ATCAGTGAAAGTTTCATGCAAAGC | GACAGAGTGACAATTACATGCCGG |
| | TAGTGGTTACACCTTCACCAGCTAT | GCCTCACAGGATATTGGGATCTCCC |
| | TGGATGAACTGGGTGCGGCAGGCC | TGAACTGGCTGCAACAGGAACCA |
| | CCCGGTCAGGGGCTTGAGTGGATG | GGAAAGGCCCCTAAGCGCCTGATA |
| | GGCATGATCCACCCATCCGACTCTG | TATGCCACATCCTCTCTTGACTCAG |
| | AGACTAGGCTGAACCAGAAGTTTA | GGGTCCCAAAGAGGTTTAGCGGCA |
| | TGGATAGAGTGACCATGACAAGAG | GTGGATCAGGTACTGAGTTCACTC |
| | ATACGTCCACTTCTACTGTCTATATG | TCACCATCTCTAGCCTGCAGCCTG |
| | GAACTGAGCAGTCTGAGATCTGAA | AGGATTTTGCAACCTACTATTGTTT |
| | GACACAGCCGTTTACTACTGTGCT | GCAATACGCTAGTTCCCCCTACACG |
| | CGCGGACTCTATGGCAATAGCTGG | TTCGGCCAGGGCACCAAAGTGGA |
| | GGCCAAGGAACATTGGTAACCGTC | AATCAAA |
| | TCTTCT | (SEQ ID NO: 60) |
| | (SEQ ID NO: 59) | |
| 3H7H21G4 | (Protein Sequence) | (Protein Sequence) |
| | QVQLVQSGAEVKKPGASVKVSCKA | DIQMTQSPSSLSASVGDRVTITCRAS |
| | SGYTFTSYWMNWVRQRPGQGLEW | QDIGISLNWYQQKPGKAPKRLIYAT |
| | IGMIHPSDSETRLNQKFMDRVTMTR | SSLDSGVPSRFSGSGSGTEFTLTISSL |
| | DTSTSTVYMELSSLRSEDTAVYYCA | QPEDFATYYCLQYASSPYTFGQGTK |
| | RGLYGNSWGQGTLVTVSS | VEIK |
| | (SEQ ID NO: 61) | (SEQ ID NO: 62) |
| | (Coding Nucleotide Sequence) | (Coding Nucleotide Sequence) |
| | CAAGTCCAGCTCGTGCAGTCTGGC | GACATCCAGATGACCCAATCTCCCT |
| | GCTGAGGTGAAAAAGCCCGGGGC | CCTCCCTGAGCGCATCCGTGGGGG |
| | CTCAGTGAAGGTTTCTTGCAAGGC | ATAGAGTGACCATAACCTGCCGGG |
| | AAGCGGCTACACCTTTACCTCCTAT | CCTCTCAGGACATCGGTATTTCTTT |
| | TGGATGAATTGGGTGCGACAGCGG | GAATTGGTATCAGCAGAAGCCCGG |
| | CCAGGCCAGGGGTTGGAGTGGATC | GAAGGCCCCTAAACGCCTGATCTA |
| | GGAATGATTCACCCTAGTGACTCA | TGCTACTTCCAGTCTGGACAGCGG |
| | GAAACTAGGCTGAACCAGAAATTC | GGTCCCGTCAAGGTTTTCAGGCAG |
| | ATGGACAGAGTCACAATGACGCGC | TGGATCAGGCACAGAGTTTACACT |
| | GATACAAGCACTAGTACAGTTTAC | CACAATTTCGAGCCTGCAGCCTGA |
| | ATGGAGCTGAGCAGCCTGAGATCG | AGATTTCGCCACTTATTACTGTCTT |
| | GAAGATACTGCCGTGTATTACTGTG | CAATACGCTAGCTCTCCATACACGT |
| | CTAGGGGACTGTATGGAAACTCTT | TCGGCCAGGGAACCAAGGTTGAG |
| | GGGGTCAAGGCACCCTTGTAACCG | ATTAAA |
| | TCTCCTCC | (SEQ ID NO: 64) |
| | (SEQ ID NO: 63) | |
| 3H7H22G4 | (Protein Sequence) | (Protein Sequence) |
| | QVQLVQSGAEVKKPGASVKVSCKA | DIQMTQSPSSLSASVGDRVTITCRAS |
| | SGYTFTSYWMNWVRQRPGQGLEW | QDIGISLNWLQQEPGKAPKRLIYAT |
| | IGMIHPSDSETRLNQKFMDRVTMTR | SSLDSGVPKRFSGSGSGTEFTLTISSL |
| | DTSTSTVYMELSSLRSEDTAVYYCA | QPEDFATYYCLQYASSPYTFGQGTK |
| | RGLYGNSWGQGTLVTVSS | VEIK |
| | (SEQ ID NO: 65) | (SEQ ID NO: 66) |
| | (Coding Nucleotide Sequence) | (Coding Nucleotide Sequence) |
| | CAAGTCCAGCTCGTGCAGTCTGGC | GACATCCAGATGACTCAGTCCCCC |
| | GCTGAGGTGAAAAAGCCCGGGGC | TCGAGCCTCTCAGCTTCTGTTGGA |
| | CTCAGTGAAGGTTTCTTGCAAGGC | GACAGAGTGACAATTACATGCCGG |
| | AAGCGGCTACACCTTTACCTCCTAT | GCCTCACAGGATATTGGGATCTCCC |
| | TGGATGAATTGGGTGCGACAGCGG | TGAACTGGCTGCAACAGGAACCA |
| | CCAGGCCAGGGGTTGGAGTGGATC | GGAAAGGCCCCTAAGCGCCTGATA |
| | GGAATGATTCACCCTAGTGACTCA | TATGCCACATCCTCTCTTGACTCAG |
| | GAAACTAGGCTGAACCAGAAATTC | GGGTCCCAAAGAGGTTTAGCGGCA |
| | ATGGACAGAGTCACAATGACGCGC | GTGGATCAGGTACTGAGTTCACTC |
| | GATACAAGCACTAGTACAGTTTAC | TCACCATCTCTAGCCTGCAGCCTG |
| | ATGGAGCTGAGCAGCCTGAGATCG | AGGATTTTGCAACCTACTATTGTTT |
| | GAAGATACTGCCGTGTATTACTGTG | GCAATACGCTAGTTCCCCCTACACG |
| | CTAGGGGACTGTATGGAAACTCTT | TTCGGCCAGGGCACCAAAGTGGA |
| | GGGGTCAAGGCACCCTTGTAACCG | AATCAAA |
| | TCTCCTCC | (SEQ ID NO: 68) |
| | (SEQ ID NO: 67) | |

### 4.4. Production and Purification of Humanized Anti-Tie2 Antibodies

To produce humanized anti-Tie2 antibodies, Expi293F™ (Gibco™) cells capable of producing recombinant proteins with high efficiency were used. Expi293F™ cells (2×10⁶ cells/ml) were cultured in Erlenmeyer flask, and plasmids encoding heavy chain and light chain were co-transfected into Expi293F™ cells with the ExpiFectamine™ 293 transfection kit (Gibco™). Cells were cultured at 37° C. under 8% CO₂ for 5 days in a shaking incubator (orbital shaker, 125 rpm). The resulting culture medium was collected and centrifuged to remove the cells. The culture supernatant containing secreted antibodies was isolated and stored at 4° C. or immediately purified using an AKTA purification system (GE Healthcare) equipped with an affinity column (Protein A agarose column, GE Healthcare). The purified antibody was concentrated by passing it through a protein centrifugal filter (Amicon®) while the solution was replaced with PBS.

### Example 5. Affinity Measurement of Humanized Anti-Tie2 Antibodies Against hTie2

The affinity of humanized anti-Tie2 antibody against hTie2 was measured using Octet® system (ForteBio) where Black 96-well plates (96 well F-type black plates, Greiner) were used. The biosensor used for affinity measurements was hydrated for 10 min before measurement with AR2G tip (ForteBio Octet®). After the hydration, humanized anti-Tie2 antibody was diluted in 10 mM sodium acetate, pH 6.0 buffer at a concentration of 10 μg/ml, fixed on AR2G biosensor, and blocked with 1M ethanolamine. The recombinant hTie2 was diluted to 50, 25, 12.5, 6.25, 3.125, and 0 nM with 1× kinetic buffer, and subjected to association for 300 sec and dissociation for 900 sec. For affinity measurement (K_{D}), association rate (K-on) and dissociation rate (K-off) were analyzed by binding curve (global) and fitted to 1:1 binding model using Octet® data analysis v9.0.0.10 program. The K_{D} values were shown in the following Table 15.

TABLE 15
Affinities of humanized 3H7 antibodies to hTie2-Ig3-FNIII(1-3)

| Antibody | Kon (1/Ms) | Koff (1/s) | K_{D} (M) |
|---|---|---|---|
| 3H7 | 5.58E+05 | 1.05E−05 | 2.69E−11 |
| 3H7H11G4 | 6.26E+04 | 4.67E−04 | 7.47E−09 |
| 3H7H12G4 | 1.04E+04 | 9.99E−04 | 9.65E−08 |
| 3H7H21G4 | 7.85E+04 | 5.17E−04 | 6.59E−09 |
| 3H7H22G4 | 1.65E+04 | 6.56E−04 | 3.97E−08 |

### Example 6. Analysis of in Vitro Biological Property of the Selected Humanized Anti-Tie2 Antibodies

### 6.1. Akt Phosphorylation

To investigate whether the humanized anti-Tie2 antibodies induce the downstream signaling of the Tie2 receptor in endothelial cells, HUVECs (Lonza™) were treated with humanized anti-Tie2 antibody. Then, the level of Akt phosphorylation, the main downstream signaling protein of Tie2 receptor, was measured by immunoblotting. To compare the degree of Akt activation, cells were treated with COMP-Ang1 (CA1) as a positive control in the experiment. Specifically, HUVEC cells (1×10⁵ cells/ml) were cultured in EGM-2 (Lonza™) at 37° C. in 60 mm culture dish. Cells of 90% confluency were incubated with EBM-2 (Lonza™) for 4 hr. The serum-starved HUVECs were treated with an anti-Tie2 antibody, and further incubated for 30 min. The cells were washed with cold PBS, treated with lysis buffer, and lysed at 4° C. for 20 min. Then, the cell lysates were prepared by centrifugation at 13000 rpm for 15 min. 5× SDS sample buffer was added to the cell lysate and the mixture was boiled at 95° C. for 5 min. Then, the mixture was subjected to SDS-PAGE and subsequent Western blotting.

To investigate the phosphorylation of Akt, the membrane was blocked with 5% skim milk-containing TBST for 1 hr at RT, and incubated with anti-phospho-Akt antibody (S473) at 4° C. for about 8 hr. The amount of phospho-Akt was visualized by enhanced chemiluminescence (ECL). Then, the membrane was incubated in a stripping buffer (Thermo Scientific) for 15 min, and then reprobed with an anti-Akt antibody to determine the amount of total Akt.

As shown in FIG. 7, Akt phosphorylation increased markedly by the treatment of humanized 3H7 antibodies. These data indicate that the humanized anti-Tie2 antibodies are able to strongly induce the activation of Akt, the main downstream signaling molecule of Tie2 receptor in endothelial cells.

### Example 7. The Effect of 3H7H12G4 in a Mouse Model of Primary Open-Angle Glaucoma

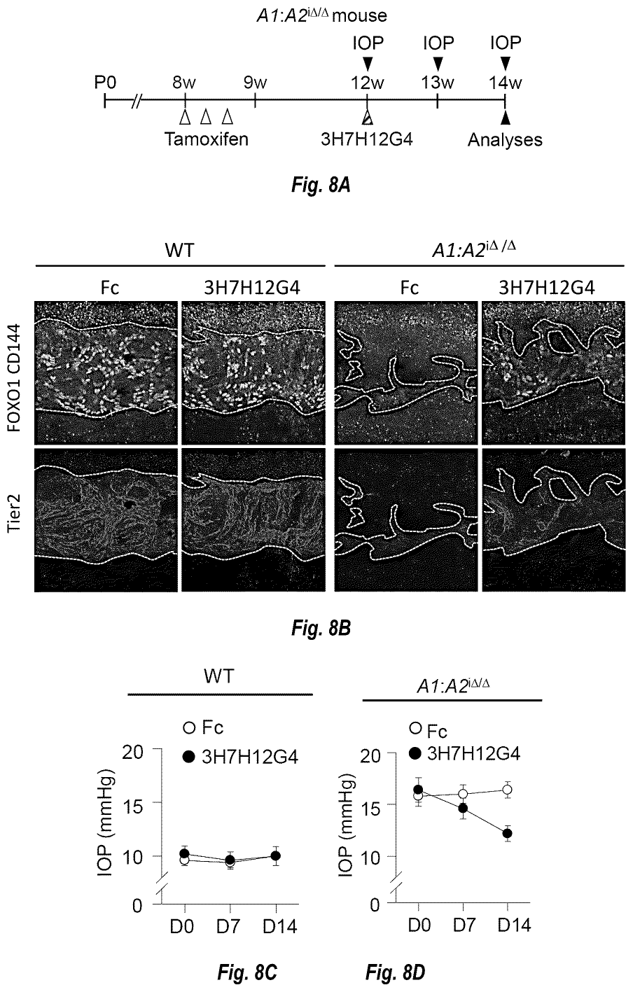

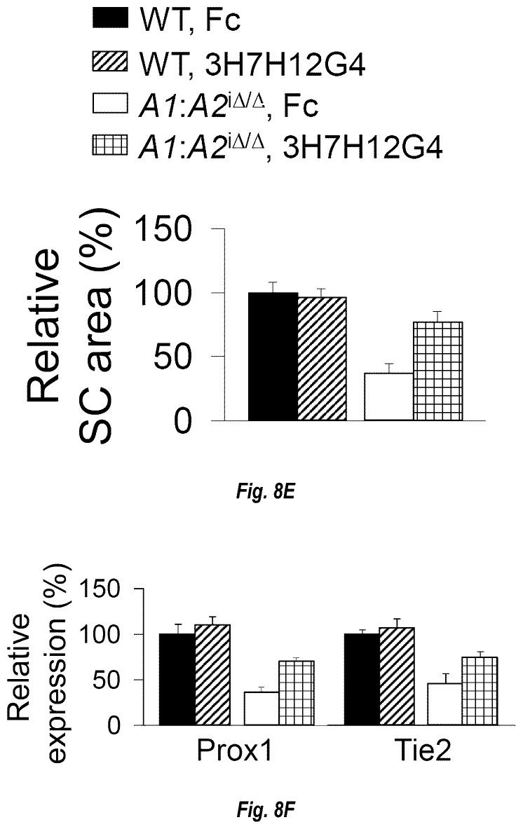

To test whether Tie2 activation by 3H7H12G4 could rescue regressed SC (Schlemm's canal) and lower IOP (intraocular pressure), we used a mouse model of primary open-angle glaucoma. The model is made by tamoxifen administration into 8-weeks-old double Angiopoietin-1/Angiopoietin-2 deficient (A1:A2^{iΔ/Δ}) mice for inducible deletion of both angiopoietin-1 and -2 genes (FIG. 8A). The intraocular administration of 3H7H12G4 (˜5 μg, one eye) and Fc (˜5 μg, contralateral eye) was performed at 12 weeks old (FIG. 8A). To intravitreally administer indicated reagents, ˜1 μl (5 mg/ml) containing 5 μg of each reagent was injected into the vitreal cavity using the Nanoliter 2000 micro-injector (World Precision Instruments) fitted with a glass capillary pipette. IOP measurements were performed with a rebound tonometer (TonoLab®) at 12, 13 and 14 weeks old (FIG. 8A). IOP was measured by placing the tip of the pressure sensor approximately ⅛ inch from the central cornea immediately after anesthetizing the mice. The digital readouts of 5 consecutive IOP measurements were acquired from the tonometer. In wild-type mice, there is no difference of IOP between 3H7H12G4-treated eyes and Fc-treated eyes (FIG. 8C). On the other hand, A1:A2^{iΔ/Δ} mice showed significant decrease of IOP in 3H7H12G4-treated eyes by 25.6% compared with that of Fc-treated eyes (FIG. 8D). CD144⁺ SC area and intensities of Prox1 and Tie2 immunostaining in CD144⁺ SC were measured at 2 weeks after 3H7H12G4 administration. Anti-CD144 antibody (1:200, BD Biosciences), anti-Prox1 antibody (1:200, ReliaTech), and anti-Tie2 antibody (1:200, R&D Systems®) were used. CD144⁺ SC area of whole-mounted cornea was calculated as a percentage of the CD144⁺ area divided by its control area. A1:A2^{iΔ/Δ} mice showed significant increase of SC area in 3H7H12G4-treated eyes by 114.8% compared with that of Fc-treated eyes (FIG. 8B, E). To quantify the relative expression of Prox1, intensities were measured in the nucleus region of CD144⁺ SC. A1:A2^{iΔ/Δ} mice showed significant increase of Prox1 intensity in 3H7H12G4-treated eyes by 88.4% compared with that of Fc-treated eyes (FIG. 8B, F). To quantify the expression of Tie2, intensities were measured in the CD144⁺ SC area. A1:A2^{iΔ/Δ}mice showed significant increase of Tie2 intensity in 3H7H12G4-treated eyes by 63.0% compared with that of Fc-treated eyes (FIG. 8B, F). Overall, these findings indicate that Tie2 activation by 3H7H12G4 rescues the impaired SC of A1:A2^{iΔ/Δ} mice.

### Example 8. CNV Regression and Vascular Leakage Suppression by Intravitreally Injected 3H7H12G4 in Laser-Induced CNV Model

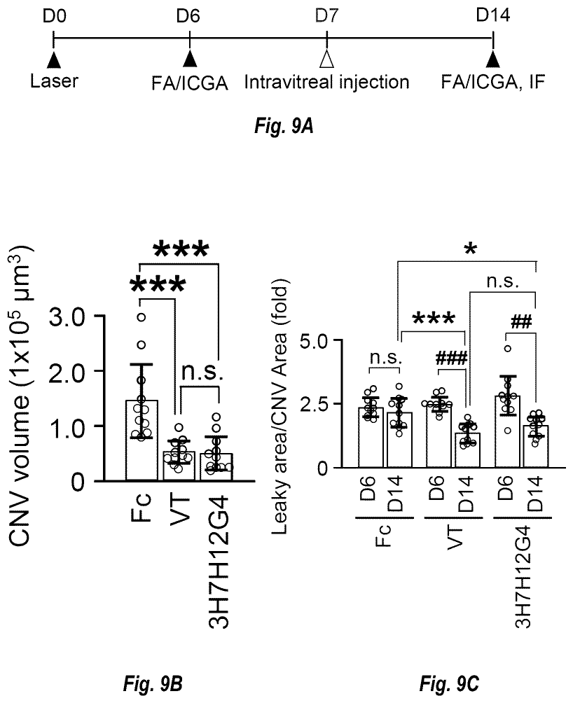

3H7H12G4 was tested for its ability to inhibit choroidal neovascularization (CNV), the hallmark of wet age-related macular degeneration (AMD) using laser-induced CNV model. After dilation of pupils with 5 mg/ml phenylephrine and 5 mg/ml tropicamide eye drops (Santen Pharmaceutical) and instillation of 0.5% proparacaine hydrochloride eye drops (Alcon) for topical anesthesia, laser photocoagulator (Lumenis Inc.) with a slit lamp delivery system was used with a glass coverslip as a contact lens to visualize the retina. Sufficient laser energy (532 nm wavelength, 250 mW power, 100 ms duration, 50 μm spot size) was delivered in 4 locations for each eye (the 3, 6, 9 and 12 o′clock positions of the posterior pole). Only burns that produced a bubble at the time of laser photocoagulation, indicating the rupture of the Bruch's membrane, were included in this study. Spots containing hemorrhage at the laser site were excluded from the analysis. To recapitulate a clinical situation, 3H7H12G4 (5 μg) was administered intravitreally to the mice at 7 days after laser photocoagulation (FIG. 9A). As a control or as for comparison, Fc or VEGF-Trap (5 μg each) was administered in a same manner to the mice. To intravitreally administer indicated reagents, ˜1 μl (5 mg/ml) containing 5 μg of each reagent was injected into the vitreal cavity using the Nanoliter 2000 micro-injector (World Precision Instruments) fitted with a glass capillary pipette. CD31⁺ CNV volumes of the retinal pigment epithelium (RPE)-choroid-sclera flat mounts were calculated using the MATLAB Image Processing Toolbox™ (Math Works) at 14 days after laser photocoagulation. Anti-CD31 antibody (1:200, Millipore®) was used for the detection of endothelial cells of CNV. VEGF-Trap effectively induced CNV regression by 64.4% compared with Fc, and 3H7H12G4 similarly induced CNV regression (65.7%) (FIG. 9B). Combined fluorescein angiography (FA) and indocyanine green angiography (ICGA) enabled us to measure vascular leakage at the neovessels around the laser injury site. Continuous-wave laser modules at 488 nm and 785 nm were used as excitation sources for fluorescein and ICG, respectively. A raster scanning pattern of excitation lasers was achieved by a scanner system consisting of a rotating polygonal mirror (MC-5; Lincoln Laser) and a galvanometer-based scanning mirror (6230H; Cambridge Technology), and delivered to the back aperture of an imaging lens. A high numerical aperture (NA) objective lens (PlanApo λ, NA 0.75; Nikon) was used as the imaging lens to provide wide-field fundus fluorescence images. Fluorescence signals detected by photomultiplier tubes (R9110; Hamamatsu Photonics) were digitized by frame grabber and reconstructed to images with size of 512×512 pixels per frame in real time. To visualize late-phase (6 min) FA and ICGA images utilizing the angiography system, 10 mg of fluorescein sodium (Alcon) and 0.15 mg of ICG (Daiichi Pharmaceutical) were administered intraperitoneally and intravenously, respectively. The imaging procedure was performed under systemic anesthesia and pupil dilation to improve the quality of images. Leaky areas from CNV were calculated as the total measured hyperfluorescent areas in FA images divided by the total measured CNV areas in ICGA images using a Java-based imaging software (ImajeJ; National Institutes of Health). Compared with Fc, both VEGF-Trap (37.0%) and 3H7H12G4 (24.6%) similarly suppressed vascular leakage (FIG. 9C). Of note, the Fc-treated group showed no significant difference in vascular leakage between 6 and 14 days after laser photocoagulation, but VEGF-Trap and 3H7H12G4 markedly reduced vascular leakage (45.6% and 42.5%, respectively) (FIG. 9C). Thus, the magnitude of the suppression of CNV and vascular leakage was quantitatively indistinguishable between VEGF-Trap and 3H7H12G4 in the mouse model of laser-induced CNV.

### Example 9. Co-Localization of 3H7H12G4 and CD31 in Endothelial Cells of CNV

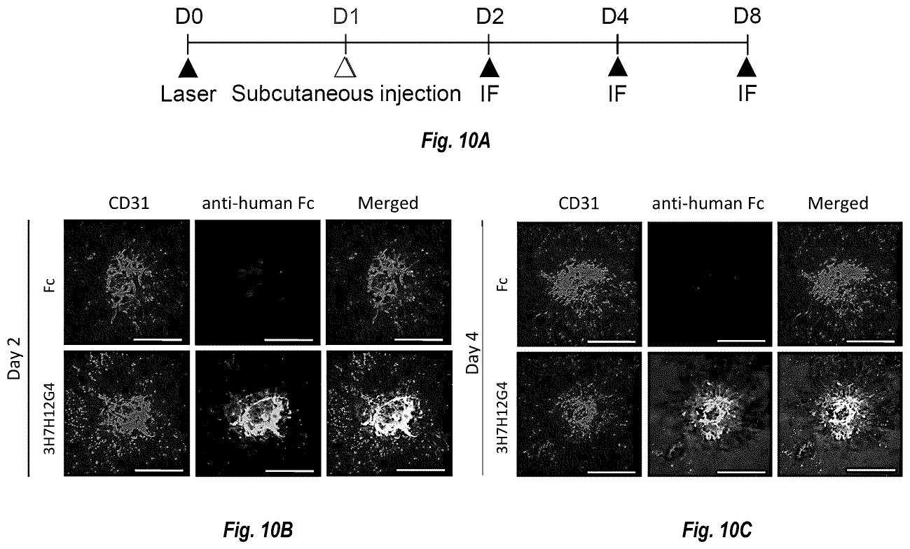

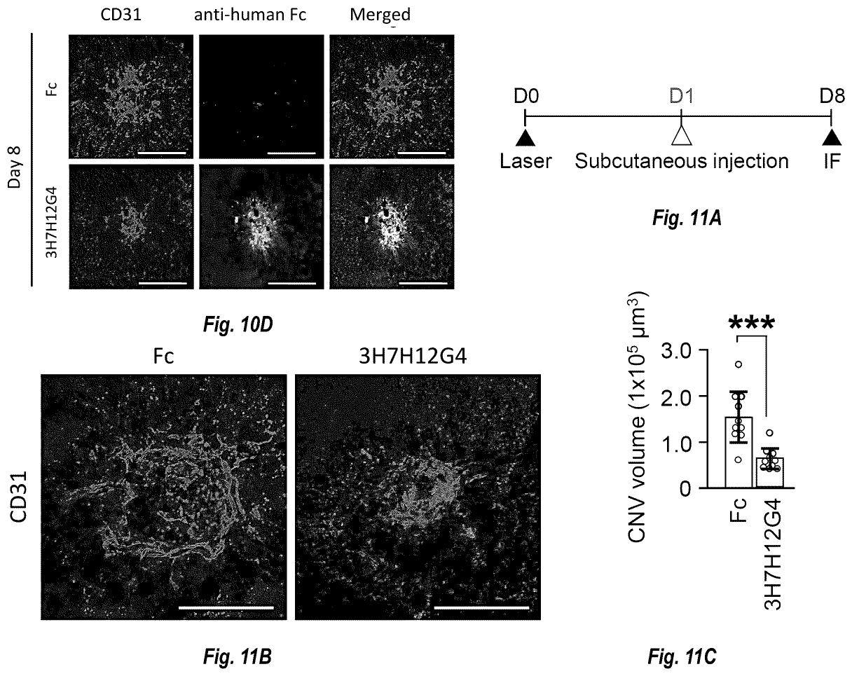

To investigate whether subcutaneously injected 3H7H12G4 can also exert the therapeutic effects on CNV, we firstly evaluated co-localization of 3H7H12G4 and CD31 in endothelial cells of CNV. The subcutaneous administration of 3H7H12G4 (25 mg/kg) was performed at 1 day after laser photocoagulation. As a control, Fc (25 mg/kg) was administered in a same manner to the mice. The co-localization of 3H7H12G4 and anti-CD31 antibody (1:200, Millipore®) in endothelial cells of CNV was directly detected by anti-human IgG antibody (1:1000, Jackson ImmunoResearch Laboratories) at 2, 4, and 8 days after laser photocoagulation (FIG. 10A). The administered 3H7H12G4 was highly detectable in the CD31⁺ endothelial cells CNV (FIG. 10B-D).

### Example 10. The CNV Inhibition Effect of Subcutaneously Injected 3H7H12G4 Antibody

To determine the effect of subcutaneously injected 3H7H12G4 in CNV inhibition, the subcutaneous administration of 3H7H12G4 (25 mg/kg) was performed at 1 day after laser photocoagulation. As a control, Fc (25 mg/kg) was administered in a same manner to the mice. Anti-CD31 antibody (1:200, Millipore®) was used for the detection of endothelial cells of CNV, and CD31⁺ CNV volumes of the RPE-choroid-sclera flat mounts were calculated using the MATLAB Image Processing Toolbox™ (MathWorks) at 8 days after laser photocoagulation (FIG. 11A). 3H7H12G4 effectively inhibited CNV formation by 69.9% compared with Fc (FIG. 11B, C), indicating that not only intravitreal injection but also subcutaneous injection of 3H7H12G4 have the inhibitory effect on CNV.

### INDUSTRIAL APPLICABILITY

The antibody or antigen-binding fragment thereof that binds to Tie2 according to the present invention is binding to Tie2 with a high affinity, maintains cross-reactivity to humans and mice, and shows the desired antigen reactivity. In addition, by inducing the Tie2 phosphorylation and the activation of the Tie2 receptor, it can be usefully used to prevent or treat angiogenic diseases of interest.

Until now, specific parts of the content in the present invention has been described in detail, and it will be obvious to those having ordinary knowledge in the art that these specific technologies are merely preferable embodiments and thus the scope of the present invention is not limited to the embodiments.

### Sequence List Free Text

The electric file attached.

1.-13. (canceled)

14. A nucleic acid encoding an anti-Tie2 antibody or antigen-binding fragment thereof which binds Tie2 Ig3-FNIII (1-3) domain comprising the sequence of SEQ ID NO:2, wherein the antibody or antigen-binding fragment thereof comprises:
a heavy chain variable region comprising heavy chain CDRH1, CDRH2, and CDRH3 comprising amino acid sequences of SEQ ID NOs: 3-5, respectively, and a light chain variable region comprising light chain CDRL1, CDRL2, and CDRL3 comprising amino acid sequences of SEQ ID NOs: 6-8, respectively;
a heavy chain variable region comprising heavy chain CDRH1, CDRH2, and CDRH3 comprising amino acid sequences of SEQ ID NOs: 13-15, respectively, and a light chain variable region comprising light chain CDRL1, CDRL2, and CDRL3 comprising amino acid sequences of SEQ ID NOs: 16-18, respectively;
a heavy chain variable region comprising heavy chain CDRH1, CDRH2, and CDRH3 comprising amino acid sequences of SEQ ID NOs: 23-25, respectively, and a light chain variable region comprising light chain CDRL1, CDRL2, and CDRL3 comprising amino acid sequences of SEQ ID NOs: 26-28, respectively;
a heavy chain variable region comprising heavy chain CDRH1, CDRH2, and CDRH3 comprising amino acid sequences of SEQ ID NOs: 33-35, respectively, and a light chain variable region comprising light chain CDRL1, CDRL2, and CDRL3 comprising amino acid sequences of SEQ ID NOs: 36-38, respectively; or
a heavy chain variable region comprising heavy chain CDRH1, CDRH2, and CDRH3 comprising amino acid sequences of SEQ ID NOs: 43-45, respectively, and a light chain variable region comprising light chain CDRL1, CDRL2, and CDRL3 comprising amino acid sequences of SEQ ID NOs: 46-48, respectively.

15. The nucleic acid of claim 14, wherein the antibody or antigen-binding fragment thereof comprises:
a heavy chain variable region comprising amino acid sequence of SEQ ID NO: 9 and a light chain variable region comprising amino acid sequence of SEQ ID NO: 11;
a heavy chain variable region comprising amino acid sequence of SEQ ID NO: 19 and a light chain variable region comprising amino acid sequence of SEQ ID NO: 21;
a heavy chain variable region comprising amino acid sequence of SEQ ID NO: 29 and a light chain variable region comprising amino acid sequence of SEQ ID NO: 31;
a heavy chain variable region comprising amino acid sequence of SEQ ID NO: 39 and a light chain variable region comprising amino acid sequence of SEQ ID NO: 41;
a heavy chain variable region comprising amino acid sequence of SEQ ID NO: 49 and a light chain variable region comprising amino acid sequence of SEQ ID NO: 51;
a heavy chain variable region comprising amino acid sequence of SEQ ID NO: 53 and a light chain variable region comprising amino acid sequence of SEQ ID NO: 54;
a heavy chain variable region comprising amino acid sequence of SEQ ID NO: 57 and a light chain variable region comprising amino acid sequence of SEQ ID NO: 58;
a heavy chain variable region comprising amino acid sequence of SEQ ID NO: 61 and a light chain variable region comprising amino acid sequence of SEQ ID NO: 62; or
a heavy chain variable region comprising amino acid sequence of SEQ ID NO: 65 and a light chain variable region comprising amino acid sequence of SEQ ID NO: 66.

16. The nucleic acid of claim 14, wherein the nucleic acid comprises:
nucleotide sequences of SEQ ID NOs: 10 and 12;
nucleotide sequences of SEQ ID NOs: 20 and 22;
nucleotide sequences of SEQ ID NOs: 30 and 32;
nucleotide sequences of SEQ ID NOs: 40 and 42;
nucleotide sequences of SEQ ID NOs: 50 and 52;
nucleotide sequences of SEQ ID NOs: 55 and 56;
nucleotide sequences of SEQ ID NOs: 59 and 60;
nucleotide sequences of SEQ ID NOs: 63 and 64; or nucleotide sequences of SEQ ID NOs: 67 and 68.

17. The nucleic acid of claim 14, wherein the antibody or antigen-binding fragment thereof comprises a heavy chain variable region comprising heavy chain CDRs comprising amino acid sequences of SEQ ID NOs: 23-25 and a light chain variable region comprising light chain CDRs comprising amino acid sequences of SEQ ID NOs: 26-28.

18. The nucleic acid of claim 15, wherein the antibody or antigen-binding fragment thereof comprises a heavy chain variable region comprising amino acid sequence of SEQ ID NO: 29 and a light chain variable region comprising amino acid sequence of SEQ ID NO: 31.

19. The nucleic acid of claim 16, wherein the nucleic acid comprises nucleotide sequences of SEQ ID NOs: 30 and 32.

20. An expression vector comprising the nucleic acid of claim 14.

21. An expression vector comprising the nucleic acid of claim 17.

22. An expression vector comprising the nucleic acid of claim 18.

23. A cell transformed with the expression vector of claim 20.

24. A cell transformed with the expression vector of claim 21.

25. A cell transformed with the expression vector of claim 22.

26. A method of manufacturing an antibody or antigen-binding fragment thereof which binds Tie2, comprising the steps of:
(a) culturing the cell of claim 23; and
(b) recovering an antibody or antigen-binding fragment thereof from the cultured cell.

27. A method of manufacturing an antibody or antigen-binding fragment thereof which binds Tie2, comprising the steps of:
(a) culturing the cell of claim 24; and
(b) recovering an antibody or antigen-binding fragment thereof from the cultured cell.

28. A method of manufacturing an antibody or antigen-binding fragment thereof which binds Tie2, comprising the steps of:
(a) culturing the cell of claim 25; and
(b) recovering an antibody or antigen-binding fragment thereof from the cultured cell.


SEQUENCE LISTING (68 sequences)

SEQ ID NO: 1 - protein, 1124 aa, synthetic construct
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 2 - protein, 390 aa, synthetic construct
TPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAP

SEQ ID NO: 3 - protein, 5 aa, synthetic construct
SYWMN

SEQ ID NO: 4 - protein, 17 aa, synthetic construct
MIHPSDSETRLNQKFKD

SEQ ID NO: 5 - protein, 6 aa, synthetic construct
GNYFDC

SEQ ID NO: 6 - protein, 11 aa, synthetic construct
RASQDIGISLN

SEQ ID NO: 7 - protein, 7 aa, synthetic construct
ATSILDS

SEQ ID NO: 8 - protein, 9 aa, synthetic construct
LQYASSPWT

SEQ ID NO: 9 - protein, 115 aa, synthetic construct
QVQLQQPGAELVRPGASVKLSCKASGYSFTSYWMNWVKQRPGQGLEWIGMIHPSDSETRLNQKFKDKATLTVDKSSSTAYLQLSSPTSEDSAVYYCARGNYFDCWGQGTTLTVSS

SEQ ID NO: 10 - DNA, 345 bp, synthetic construct
  source 1..345: mol_type = other DNA
caggtccaactgcagcagcctggggctgagctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacttgcaactcagcagcccgacatctgaggactctgcggtctattactgtgcaagggggaactactttgactgctggggccaaggcaccactctcacagtctcctca

SEQ ID NO: 11 - protein, 107 aa, synthetic construct
DIQMTQSPSSLSASLGERVSLTCRASQDIGISLNWLQQEPDGTIKRLIYATSILDSGVPKRFSGSRSGSDYSLTISSLESEDFVDYYCLQYASSPWTFGGGTKLEIK

SEQ ID NO: 12 - DNA, 321 bp, synthetic construct
  source 1..321: mol_type = other DNA
gacatccagatgacccagtctccatcctccttatctgcctctctgggagaaagggtcagtctcacttgtcgggcaagtcaggacattggtattagcttaaactggcttcagcaggaaccagatggaactattaaacgcctgatctacgccacatccattttagattctggtgtccccaaaaggttcagtggcagtaggtctgggtcagattattctctcaccatcagcagccttgagtctgaagattttgtagactattactgtctacaatatgctagttctccgtggacgttcggtggaggcaccaagctggaaatcaaa

SEQ ID NO: 13 - protein, 5 aa, synthetic construct
SYWMN

SEQ ID NO: 14 - protein, 17 aa, synthetic construct
MIHPSDSETRLNQKFKD

SEQ ID NO: 15 - protein, 6 aa, synthetic construct
GYYFGY

SEQ ID NO: 16 - protein, 11 aa, synthetic construct
RASQDIGISLN

SEQ ID NO: 17 - protein, 7 aa, synthetic construct
ATSNLDS

SEQ ID NO: 18 - protein, 9 aa, synthetic construct
LQYASSPPT

SEQ ID NO: 19 - protein, 115 aa, synthetic construct
QVQLQQPGADLVRPGASVKLSCKASGYSFTSYWMNWVKQRPGQGLEWIGMIHPSDSETRLNQKFKDKATLTVDKSSSTAYMQLSSPTSEDSAVYYCAKGYYFGYWGQGTTLTVSS

SEQ ID NO: 20 - DNA, 345 bp, synthetic construct
  source 1..345: mol_type = other DNA
caggtccaactgcagcagcctggggctgacctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacatgcaactcagcagcccgacatctgaggactctgcggtctattactgtgcaaaggggtactactttggctactggggccaaggcaccactctcacagtctcctca

SEQ ID NO: 21 - protein, 107 aa, synthetic construct
DIQMTQSPSSLSASLGERVSLTCRASQDIGISLNWLQQEPDGTIKRLIYATSNLDSGVPKRFSGSRSGSDYSLTISSLESEDFVDYYCLQYASSPPTFGGGTKLEIK

SEQ ID NO: 22 - DNA, 321 bp, synthetic construct
  source 1..321: mol_type = other DNA
gacatccagatgacccagtctccatcctccttatctgcctctctgggagaaagagtcagtctcacttgtcgggcaagtcaggacattggtattagtttaaactggcttcagcaggaaccagatggaactattaaacgcctgatctacgccacatccaatttagattctggtgtccccaaaaggttcagtggcagtaggtctgggtcagattattctctcaccatcagcagccttgagtctgaagattttgtagactattactgtctacaatatgctagttctcctccgacgttcggtggaggcaccaagctggaaatcaaa

SEQ ID NO: 23 - protein, 5 aa, synthetic construct
SYWMN

SEQ ID NO: 24 - protein, 17 aa, synthetic construct
MIHPSDSETRLNQKFMD

SEQ ID NO: 25 - protein, 6 aa, synthetic construct
GLYGNS

SEQ ID NO: 26 - protein, 11 aa, synthetic construct
RASQDIGISLN

SEQ ID NO: 27 - protein, 7 aa, synthetic construct
ATSSLDS

SEQ ID NO: 28 - protein, 9 aa, synthetic construct
LQYASSPYT

SEQ ID NO: 29 - protein, 115 aa, synthetic construct
QVQLQQPGAELVRPGASVKLSCKASGYSFTSYWMNWVKQRPGQGLEWIGMIHPSDSETRLNQKFMDKATLTVDKSSSTAYMQLSSPTSEDSAVYYCARGLYGNSWGQGTLVTVSA

SEQ ID NO: 30 - DNA, 345 bp, synthetic construct
  source 1..345: mol_type = other DNA
caggtccaactgcagcagcctggggctgagctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaaatcagaagttcatggacaaggccacattgactgtagacaaatcctccagcacagcctacatgcagctcagcagcccgacatctgaggactctgcggtctattactgtgctcgtggcctctatggtaactcttggggccaagggactctggtcactgtctctgca

SEQ ID NO: 31 - protein, 107 aa, synthetic construct
DIQMTQSPSSLSASLGERVSLTCRASQDIGISLNWLQQEPDGTIKRLIYATSSLDSGVPKRFSGSRSGSDYSLTISSLESEDFVDYYCLQYASSPYTFGGGTKLEIK

SEQ ID NO: 32 - DNA, 321 bp, synthetic construct
  source 1..321: mol_type = other DNA
gacatccagatgacccagtctccatcctccttatctgcctctctgggagaaagagtcagtctcacttgtcgggcaagtcaggacattggtattagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtacacgttcggaggggggaccaagctggaaataaaa

SEQ ID NO: 33 - protein, 5 aa, synthetic construct
SYWMN

SEQ ID NO: 34 - protein, 17 aa, synthetic construct
MIHPSDSETRLNQKFKD

SEQ ID NO: 35 - protein, 6 aa, synthetic construct
GYYFDY

SEQ ID NO: 36 - protein, 11 aa, synthetic construct
RASQDIGISLN

SEQ ID NO: 37 - protein, 7 aa, synthetic construct
ATSSLDS

SEQ ID NO: 38 - protein, 9 aa, synthetic construct
LQYVSSPWT

SEQ ID NO: 39 - protein, 115 aa, synthetic construct
QVQLQQPGAELVRPGASVKLSCKASGYSFTSYWMNWVKQRPGQGLEWIGMIHPSDSETRLNQKFKDKATLTVDKSSSTAYMQLSSPTSEDSAVYYCARGYYFDYWGQGTTLTVSS

SEQ ID NO: 40 - DNA, 345 bp, synthetic construct
  source 1..345: mol_type = other DNA
caggtccaactgcagcagcctggggctgagctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacatgcaactcagcagcccgacatctgaggactctgcggtctattactgtgcaagaggctactactttgactactggggccaaggcaccactctcacagtctcctca

SEQ ID NO: 41 - protein, 107 aa, synthetic construct
DIQMTQSPSSLSASLGERVSLTCRASQDIGISLNWLQQEPDGTIKRLIYATSSLDSGVPKRFTGSRSGSDYSLTISSLESEDFVDYYCLQYVSSPWTFGGGTKLEIK

SEQ ID NO: 42 - DNA, 321 bp, synthetic construct
  source 1..321: mol_type = other DNA
gacatccagatgacccagtctccatcctccttatctgcctctctgggagaaagagtcagtctcacttgtcgggcaagtcaggacattggtattagcttaaactggcttcagcaggaaccagatggaactattaaacgcctgatctacgccacatccagtttagattctggtgtccccaagaggttcactggcagtaggtctgggtcagattattctctcaccatcagcagccttgagtctgaagattttgtagactattactgtctacaatatgttagttctccgtggacgttcggtggaggcaccaagctggaaatcaaa

SEQ ID NO: 43 - protein, 5 aa, synthetic construct
SYWMN

SEQ ID NO: 44 - protein, 17 aa, synthetic construct
MIHPSDSETRLNQKFKD

SEQ ID NO: 45 - protein, 7 aa, synthetic construct
LTIYFDY

SEQ ID NO: 46 - protein, 11 aa, synthetic construct
RASQDIGISLN

SEQ ID NO: 47 - protein, 7 aa, synthetic construct
ATSSLDS

SEQ ID NO: 48 - protein, 9 aa, synthetic construct
LQYASSPYT

SEQ ID NO: 49 - protein, 116 aa, synthetic construct
QVQLQQPGADLVRPGASVTLSCKASGYSFTSYWMNWVKQRPGQGLEWIGMIHPSDSETRLNQKFKDKATLTVDKSSSTAYMQLRSPTSEDSAVYYCAGLTIYFDYWGQGTTLTVSS

SEQ ID NO: 50 - DNA, 348 bp, synthetic construct
  source 1..348: mol_type = other DNA
caggtccaactacagcagcctggggctgacctggtgaggcctggagcttcagtgacgctgtcctgcaaggcttctggctactccttcaccagctactggatgaactgggtgaagcagaggcctggacaaggcctggagtggattggcatgattcatccttccgatagtgaaactaggttaaatcagaagttcaaggacaaggccacattgactgtagacaaatcctccagcacagcctacatgcaactccgcagcccgacatctgaggactctgcggtctattactgtgcaggcctaactatttactttgactattggggccaaggcaccactctcacagtctcctca

SEQ ID NO: 51 - protein, 107 aa, synthetic construct; identical to SEQ ID NO: 31

SEQ ID NO: 52 - DNA, 321 bp, synthetic construct; identical to SEQ ID NO: 32
  source 1..321: mol_type = other DNA

SEQ ID NO: 53 - protein, 115 aa, synthetic construct
QVQLVQSGAEVKKPGASVKVSCKASGYTFTSYWMNWVRQAPGQGLEWMGMIHPSDSETRLNQKFMDRVTMTRDTSTSTVYMELSSLRSEDTAVYYCARGLYGNSWGQGTLVTVSS

SEQ ID NO: 54 - protein, 107 aa, synthetic construct
DIQMTQSPSSLSASVGDRVTITCRASQDIGISLNWYQQKPGKAPKRLIYATSSLDSGVPSRFSGSGSGTEFTLTISSLQPEDFATYYCLQYASSPYTFGQGTKVEIK

SEQ ID NO: 55 - DNA, 345 bp, synthetic construct
  source 1..345: mol_type = other DNA
caggtgcagctggtccaatccggggctgaggtgaagaagcctggagcatcagtgaaagtttcatgcaaagctagtggttacaccttcaccagctattggatgaactgggtgcggcaggcccccggtcaggggcttgagtggatgggcatgatccacccatccgactctgagactaggctgaaccagaagtttatggatagagtgaccatgacaagagatacgtccacttctactgtctatatggaactgagcagtctgagatctgaagacacagccgtttactactgtgctcgcggactctatggcaatagctggggccaaggaacattggtaaccgtctcttct

SEQ ID NO: 56 - DNA, 321 bp, synthetic construct
  source 1..321: mol_type = other DNA
gacatccagatgacccaatctccctcctccctgagcgcatccgtgggggatagagtgaccataacctgccgggcctctcaggacatcggtatttctttgaattggtatcagcagaagcccgggaaggcccctaaacgcctgatctatgctacttccagtctggacagcggggtcccgtcaaggttttcaggcagtggatcaggcacagagtttacactcacaatttcgagcctgcagcctgaagatttcgccacttattactgtcttcaatacgctagctctccatacacgttcggccagggaaccaaggttgagattaaa

SEQ ID NO: 57 - protein, 115 aa, synthetic construct; identical to SEQ ID NO: 53

SEQ ID NO: 58 - protein, 107 aa, synthetic construct
DIQMTQSPSSLSASVGDRVTITCRASQDIGISLNWLQQEPGKAPKRLIYATSSLDSGVPKRFSGSGSGTEFTLTISSLQPEDFATYYCLQYASSPYTFGQGTKVEIK

SEQ ID NO: 59 - DNA, 345 bp, synthetic construct; identical to SEQ ID NO: 55
  source 1..345: mol_type = other DNA

SEQ ID NO: 60 - DNA, 321 bp, synthetic construct
  source 1..321: mol_type = other DNA
gacatccagatgactcagtccccctcgagcctctcagcttctgttggagacagagtgacaattacatgccgggcctcacaggatattgggatctccctgaactggctgcaacaggaaccaggaaaggcccctaagcgcctgatatatgccacatcctctcttgactcaggggtcccaaagaggtttagcggcagtggatcaggtactgagttcactctcaccatctctagcctgcagcctgaggattttgcaacctactattgtttgcaatacgctagttccccctacacgttcggccagggcaccaaagtggaaatcaaa

SEQ ID NO: 61 - protein, 115 aa, synthetic construct
QVQLVQSGAEVKKPGASVKVSCKASGYTFTSYWMNWVRQRPGQGLEWIGMIHPSDSETRLNQKFMDRVTMTRDTSTSTVYMELSSLRSEDTAVYYCARGLYGNSWGQGTLVTVSS

SEQ ID NO: 62 - protein, 107 aa, synthetic construct; identical to SEQ ID NO: 54

SEQ ID NO: 63 - DNA, 345 bp, synthetic construct
  source 1..345: mol_type = other DNA
caagtccagctcgtgcagtctggcgctgaggtgaaaaagcccggggcctcagtgaaggtttcttgcaaggcaagcggctacacctttacctcctattggatgaattgggtgcgacagcggccaggccaggggttggagtggatcggaatgattcaccctagtgactcagaaactaggctgaaccagaaattcatggacagagtcacaatgacgcgcgatacaagcactagtacagtttacatggagctgagcagcctgagatcggaagatactgccgtgtattactgtgctaggggactgtatggaaactcttggggtcaaggcacccttgtaaccgtctcctcc

SEQ ID NO: 64 - DNA, 321 bp, synthetic construct; identical to SEQ ID NO: 56
  source 1..321: mol_type = other DNA

SEQ ID NO: 65 - protein, 115 aa, synthetic construct; identical to SEQ ID NO: 61

SEQ ID NO: 66 - protein, 107 aa, synthetic construct; identical to SEQ ID NO: 58

SEQ ID NO: 67 - DNA, 345 bp, synthetic construct; identical to SEQ ID NO: 63
  source 1..345: mol_type = other DNA

SEQ ID NO: 68 - DNA, 321 bp, synthetic construct; identical to SEQ ID NO: 60
  source 1..321: mol_type = other DNA